SEQUENCE LISTING (51 sequences)

SEQ ID NO: 1 - DNA, 22061 bp, Artificial Sequence
  Synthetic
ggaactccatatatgggctatgaactaatgaccccgtaattgattactattaataactagtcaataatcaatgtcgagggcgcgccgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggcggatcccatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcggatccgcggccgctcagttagcctcccccatctcccgggcaaacgtgcgcgccaggtcgcatatcgtcggtatggagccgggggtggtgacgtgggtctggaccatcccggaggtaagttgcagcagggcgtcccggcagccggcgggcgattggtcgtaatccaggataaagacgtgcatggaacggaggcgtttggccaagacgtccaaggcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccagtcctcccgccacgacccgccgcactgcagataccgcaccgtattggcaagtagcccgtaaacgcggcgaatcgcagccagcatagccaggtccagccgctcgccggggcgctggcgtttggccaggcggtcgatgtgtctgtcctccggaagggccccaagcacgatgttggtgccgggcaaggtcggcgggatgagggccacgaacgccagcacggcctggggggtcatgctgcccataaggtaccgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccgggcggacttccgtggcttcttgctgccggcgagggcgcaacgccgtacgtcggttgctatggccgcgagaacgcgcagcctggtcgaacgcagacgcgtgttgatggccggggtacgaagccatattaccctgttatccctagaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgacccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagcgctgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttacgccttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcggccgctattacgcacaagagttccgtagctgttcaagtttgtgtttcaactgttctcgtcgtttccgcaacaagtcctcttcagaaatgagcttttgctcctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgaggacctggggctggtgcattttcggttgttgctgatctgtctcaggactctgacactgtccaacttgaccctcttggcagcaggatagtccttccgagtggagggaggcgctgcgtagttgtgctgatgtgtggagacgtggcacctcttgaggaccagtgggctgtgaggaggtttgctgtggcctccagcagaaggtgatccagactctgaccttttgccaggagcctgcctcttttccacagaaacaacatcgatttcttcctcatcttcttgttcctcctcagagtcgctgctggtggtgggcggtgtctcctcatggagcaccaggggctcggggctgccctgcggggaggactccgtcgaggagagcagagaatccgaggacggagagaaggcgctggagtcttgcgaggcgcaggacttgggcgagctgctgtcgttgagagggtaggggaagaccaccgaggggtcgatgcactctgaggcggcggcgctcagatcctgcaggtacaagctggaggtggagcagacgctgtggccgcgggcggggttcgggctgccgctgtctttgcgcgcagcctggtaggaggccagcttctctgagacgagcttggcggcggccgagaagccgctccacatacagtcctggatgatgatgtttttgatgaaggtctcgtcgtccgggtcgcagatgaaactctggttcaccatgtctcctcccagcagctcggtcaccatctccagctggtcggccgtggagaagctcccgccaccgccgtcgttgtctccccgaagggagaagggtgtgaccgcaacgtaggagggcgagcagagcccggagcggcggctaggggacaggggcggggtgggcagcagctcgaatttcttccagatatcctcgctgggcgccgggggctgcagctcgctctgctgctgctgctggtagaagttctcctcctcgtcgcagtagaaatacggctgcaccgagtcgtagtcgaggtcatagttcctgttggtgaagctaacgttgaggggcataggaccggggttactttcaacatcgccagcgagtttcaacaaagcgtagttagtacattgcccactacccatgtgtgagaggggcagtgtgccgttaatggccgtgccgggcaccgggccgctctggtagtgctgggacatgtgaagtctgctgggggcggcgggttccggcacctcggcgccggggagatacatgctgatcatgtcccggaggtccccggcctggcagggcgccctggagtgggaggaagaggtaaccacaggggggctggagctggcctcggacttgaccaccgaacccatggagccaagagccatgccaggggtgccctgctgcgagtaggacatgctgtaggtgggcgagccgttcatgtaggtctgcgagctggtcatggagttgtactgcagggcgctcacgtcgtagcggtgcatgggctgcatctgcgctgcgccgtgcgcattgaggcccgggtgctgcgggtagcccagctggtcctgcatcatgctgtagctgccgttgctccagccgttcatgtgcgcgtaactgtccatgcgctggttcacgcccgcgcccaggccggcgcccaccccgaccccgctcgccatgctattgccgccgggggccagcagcccgccgggcagcgtgtacttatccttcttcatgagcgtcttggttttccgccggggccggtatttataatccgggtgctccttcatgtgcagcgctcgcagccgcttagcctcgtcgatgaacggccgcttctccgtctccgacaaaagtttccactcggcgcccaggcgcttgctgatctccgagttgtgcatcttggggttctcctgggccatcttgcgccgctgcccgcgggaccacaccatgaaggcattcatgggccgcttgacgcggtccgggctgtttttctggttgccgccggccgccgccgcggtggagttgccgccgccgccccccgaagtttgctgcgggcccggcggcttcagctccgtctccatcatgttgtacatggggccggggttctcctccacgtcgccgcaggtcagcaggctgcccctgccctcgccgctgccaaaatgcctcttcatgtgtaaggcgaggtggtccgacctggaaaatgctcggtcgcatttttggcactggaacgggcggtgccccgtgtgtttacggtagtgcctggtcagttcatctgagcgggcgaatttccatccacagccgtcccagtcacagtggtaaggtttctcacctgtgtgggttcgcaggtgtgccttgagatgggaactctttgtgtaggttttgccgcagcccgcgtaatcacaagtgtgggtggcggtccttttccggggccacgatcgtcttcccctctttggcttgggctcctctggcatgcaggaaccgggtggcatgagctcttggtaatggagcggcgggacttgcggctgcatctgatcgggcaggaaggatgggtaattgggccccgggtggggatggaagccgggaggaagcggcagggcagggtgacagtccctgctgctcagcacttcctcaagacccagggtcggggtagtcctgctggggagctgccgccccagggggaagtcgtgtgcagccggccggtggccattgctgagagggggtccagcgcccaagtgggtgcacgaagagaccgcctcctgcttgatcttggggcacgtgcgcggcggcccgccgttgtagggcgccaccaccaccgggtggctgccgtcagggctgcctttgctgacgctgatgaccgacgggctgccgtactcgctgccaggggcgctcagcgacgccttcagcacgaacttgcccatcagcccgccacctggcggctgcggctgctgcggcggaatgtacaccgggtccaattctggccgcaggagctcggccacgaagccgcccgaggggctcacgtcgttgatgtccgccaggttgaagggagccgtcggagggggagcggactccctgccatagaggaggcctccgcccgtgccgcccggcgccacgcccgggtcgttcccggcccggatcggataggtgaagctgcaggtggagggcgcgctggcagggccgctgctcgacggcgacgacgaagaggaggctgacgctgacgaggacacggtggcggccactgactccggaggatgggtcagcgaattggagagaataaagtccaggtccaggagatcgttgaactcctcggtctctctccgaggtaggggcgccaggttgctaccgccgcaagccgcaccggctccgccgctctccaggtctgtggccacggtcgccgccgccaggtcataggggcggccgggaagcactgggggaagtcgcttcatgtgggagagctcctcccgccagcggttattcggggcacctgcttgacgcagtgtcttctcccttcccgccgggccagacgcgaacgtggagaaagatgggagcagcgcgtcgctgacagccatgggccctgggttggactccacgtctcccgccaacttgagaaggtcaaaattcaaagtctgtttcacgccacttccgtttgaatgcatgggagagcccagagtggtgacggagacagggggaaaggcttccccctcagggaaagggaccgaggagtacagtgcagtgaagtgagggctcccatagcctggggtaccaaaatggggccctggggccagaggaaaggacactggtccccctgagaaaggagacccagcagcctcaaaatcctctcgttgtgcatagtcgctgcttgatcgcttgcccttctggcgccggttacagaaccacactcggaccacatccttctcgagcccaagctgctgggcgatgtggctgatctgctgcagtgtgggtttcgggcactgcaggaacaaattctccaggttgcctctcactcggttctcgatactggttcgctttctctttcgggcctgcacgagggtttctgctttgcatatctcctgaagattttcattgttgtcagcttcctccacccacttctgcagcaagggccgcagcttacacatgttcttgaagctaagctgcagagcctcaaagcggcagatggtcgtttggctgaataccttcccaaatagaacccccagggtgagccccacatcggcctgtgtatatcccagggtgatcctcttctgcttcaggagcttggcaaattgctcgagttctttctgcagagctttgatgtcctgggactcctccgggttttgctccagcttctccttctccagcttcacggcaccaggggtgacggtgcagggctccggggaggccccatcggagttgctctccaccccgactcctgcttcgccctcaggctgagaggtctccaagccgccttggggcactagccccactccaacctggggcccacagtacgccatccccccacagaactcatacggcggggggcatgggggaatcccccacacctcagagcctggcccaacccccggcccgattcctggccctccaggagggccttggaagcttagccaggtccgaggatcaacccagcccggctccggcccccctggcccatcacctccaccacctggagggggcgagaaggcgaaatccgaagccaggtgtcccgccatggtggcgctagcttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgattaattaatcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgagcgattaaacgcgccaactagaggatcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctcgtttatcttatcaattctgtgcctccgggagcagggtagggctgtggatttcttcttcttcctctcgaaagtaggttggctttccctgtgcactagggccctgctgggccttcagcggacatgaggctaccatatggctgatgctctggcagaagtggcacttcttgggctggggtggcagcttgcattccttggcatgatgatctagacctccacagttgtagcacctgtctccttttgatctgcgcttctgcatgctctttccttttggccgcctctcactcccaatacagaatactccaccaggtccggtgacacggatggattccagacccttggctgacttcttaaaggtgaactccactgcctcaccctccttcaagctccggaacccttccatgtgcagcttactctggtgcacaaagacatccactggggggtcgagcgcgaccccggcgcgggcggtcatggacaggaagccgaaccccatgcgcacgttgaaccacttacagatgcccgcaccgtgcagcagctgaggctcgtccgccgcccgggccgcgtcctccggcgcctcctcgggcgcctcttctgccgccttggcgcagccacctgcaaactgctggttggacacggagcccatgggccctgggttggactccacgtctcccgccaacttgagaaggtcaaaattcaaagtctgtttcacgccacttcccacgtcttcaggttgcatgttcatggagtagtttaggaataaatccatggtttgtggagtactaaaatacctagtggtctgctgtattacattaaggccttccccagcagcttccaaggcagcctccaagtcactggcaggagaatttggctggaactgcatgcaggactgcagagattcctctccacagttatagaagggactgttccaggcctgattgttccaggattgggtgcaccaggtctgagtgttccaggagtggttgctccaggactggatgttctgggtctggttgctccaggttgaattgttccaggtctggttgctccacattggaaggttcccagtcgggttcaccaggcatccctggtggtaggaagagtaaaggctggggtaggtaggtgctgaggccttctgcgtcacaccattgctattcttcggccagttgtttttctgccacctcttagatttcattctctggttctggaaccaggtcttcacctgtttgtagctgaggttcaggatgttggagagttcttgcatctgctggaggctgaggtatttctgtctctgaaatctatcattgagtacacacagctgggtggaagagaacacagttctggtcttctgtttcttgaccgggaccttgtcttccttttttgcgacactcttctctgcagaagtgggttgtttgcctttgggactggtggaagaatcagggctgtcctgaataagcagatccatggaggaaggaagaggagagacagtctccgtgtgaggcatctcagcagaagacatttgcaaggatggatagttttcttcaggcccacaaatcacaggcataggtgaagattctttacagtcggatgcttcaaagcaaggcaagctttggggacaagctggatccacactcatggtggcttaatcgcgaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgacccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgc

SEQ ID NO: 2 - DNA, 25064 bp, Artificial Sequence
  Synthetic
ggaactccatatatgggctatgaactaatgaccccgtaattgattactattaataactagtcaataatcaatgtcgagggcgcgccgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgcggccgcgtcgacatttaaatttatgctcttttggcatgcaacatttcaatgagaaggttattatagggcacatccccgttcaggtgcttgtagtagaggtattcttcagcctgcatactgatggcccggatttcgggtagtcgaagaagtagctgtccaaatttctctgtctgctgcgggtagttacacattgtgtagtccagcagggcggcattgacttgttcctggacaccttctaccagctggaagttttcaaggtttttgacatctaaactaaagagcaccaagaatttcagacatacgaactctcgttgatcaaactggagagaacgaagttttgccactaactcctgtgcatgactcatgaggttgttgagggtggctccggcttgtgatgctattatggaatagtccacttgttgcccagtaaccaggaagatggatccttcctttccatgtaccacttgtcggtaaatgtggtcgaggattaagagctcactccagcagttctgaagcagcttcatttggtcatcaaccttaagttctctgaagaagatactactcctggcccactcgacaatggagaagagagtttgatctgccattttgcacataagcccaaaggtgctcagcttttcgtgcttgcttcggttagcctgctcttgctgcaaataggccatgattttagcctggacttgaggctcatctggctcacacttcaaaagttccagtatcagatgtgggatgcttgctggagagctcgtctggtaactatccatatatgaatagcccattatggactcgggtgagctggtataggggtctgggtactcagacttgatggcccggctaggaaagtggccatatgtttggtaaccttgcaggctgccgtgagggggcattgtcatgctaatgggggatgttacaaagggacttctgtcatagtctgtaggaggcaaggcagcatggttcagaggtaggcctttggaggcagagtggatgttttgaattgcagaggaaatggtcaggtcagagggcatagcttggatcacctgagacatggcttctagcttaagtccattggctcggatgagggcttttttctgttgcttcagggccctgtctctcttgtacattggcccaaacttattccttcctccacgcattcggtcggcccttacagcttctagcttcattccaacacttagacatttttgaaaacgacagtaaggacaacgctttctctgtgttttgtcaatttggcagttctggttttctatacatgtgtaccttttattattttggactgttcgcttaaaaaatcccttgcagctttcacaggtgaggagcccataatggtacccagacactttatctccacacacgggacaaagctcttccagatcttcatcataggagtaattcaccattttaaattgagacacaataggtgtaagtccgtgctttaaagactcttgtaaatccccagtatctgaattagaagacatggtggcgatcgctagcttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgattaatcgccgcgccgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggcggatcccatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcggatccgcggccgctcagttagcctcccccatctcccgggcaaacgtgcgcgccaggtcgcatatcgtcggtatggagccgggggtggtgacgtgggtctggaccatcccggaggtaagttgcagcagggcgtcccggcagccggcgggcgattggtcgtaatccaggataaagacgtgcatggaacggaggcgtttggccaagacgtccaaggcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccagtcctcccgccacgacccgccgcactgcagataccgcaccgtattggcaagtagcccgtaaacgcggcgaatcgcagccagcatagccaggtccagccgctcgccggggcgctggcgtttggccaggcggtcgatgtgtctgtcctccggaagggccccaagcacgatgttggtgccgggcaaggtcggcgggatgagggccacgaacgccagcacggcctggggggtcatgctgcccataaggtaccgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccgggcggacttccgtggcttcttgctgccggcgagggcgcaacgccgtacgtcggttgctatggccgcgagaacgcgcagcctggtcgaacgcagacgcgtgttgatggccggggtacgaagccatattaccctgttatccctagaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgacccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagcgctgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttacgccttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcggccgctattacgcacaagagttccgtagctgttcaagtttgtgtttcaactgttctcgtcgtttccgcaacaagtcctcttcagaaatgagcttttgctcctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgaggacctggggctggtgcattttcggttgttgctgatctgtctcaggactctgacactgtccaacttgaccctcttggcagcaggatagtccttccgagtggagggaggcgctgcgtagttgtgctgatgtgtggagacgtggcacctcttgaggaccagtgggctgtgaggaggtttgctgtggcctccagcagaaggtgatccagactctgaccttttgccaggagcctgcctcttttccacagaaacaacatcgatttcttcctcatcttcttgttcctcctcagagtcgctgctggtggtgggcggtgtctcctcatggagcaccaggggctcggggctgccctgcggggaggactccgtcgaggagagcagagaatccgaggacggagagaaggcgctggagtcttgcgaggcgcaggacttgggcgagctgctgtcgttgagagggtaggggaagaccaccgaggggtcgatgcactctgaggcggcggcgctcagatcctgcaggtacaagctggaggtggagcagacgctgtggccgcgggcggggttcgggctgccgctgtctttgcgcgcagcctggtaggaggccagcttctctgagacgagcttggcggcggccgagaagccgctccacatacagtcctggatgatgatgtttttgatgaaggtctcgtcgtccgggtcgcagatgaaactctggttcaccatgtctcctcccagcagctcggtcaccatctccagctggtcggccgtggagaagctcccgccaccgccgtcgttgtctccccgaagggagaagggtgtgaccgcaacgtaggagggcgagcagagcccggagcggcggctaggggacaggggcggggtgggcagcagctcgaatttcttccagatatcctcgctgggcgccgggggctgcagctcgctctgctgctgctgctggtagaagttctcctcctcgtcgcagtagaaatacggctgcaccgagtcgtagtcgaggtcatagttcctgttggtgaagctaacgttgaggggcataggaccggggttactttcaacatcgccagcgagtttcaacaaagcgtagttagtacattgcccactacccatgtgtgagaggggcagtgtgccgttaatggccgtgccgggcaccgggccgctctggtagtgctgggacatgtgaagtctgctgggggcggcgggttccggcacctcggcgccggggagatacatgctgatcatgtcccggaggtccccggcctggcagggcgccctggagtgggaggaagaggtaaccacaggggggctggagctggcctcggacttgaccaccgaacccatggagccaagagccatgccaggggtgccctgctgcgagtaggacatgctgtaggtgggcgagccgttcatgtaggtctgcgagctggtcatggagttgtactgcagggcgctcacgtcgtagcggtgcatgggctgcatctgcgctgcgccgtgcgcattgaggcccgggtgctgcgggtagcccagctggtcctgcatcatgctgtagctgccgttgctccagccgttcatgtgcgcgtaactgtccatgcgctggttcacgcccgcgcccaggccggcgcccaccccgaccccgctcgccatgctattgccgccgggggccagcagcccgccgggcagcgtgtacttatccttcttcatgagcgtcttggttttccgccggggccggtatttataatccgggtgctccttcatgtgcagcgctcgcagccgcttagcctcgtcgatgaacggccgcttctccgtctccgacaaaagtttccactcggcgcccaggcgcttgctgatctccgagttgtgcatcttggggttctcctgggccatcttgcgccgctgcccgcgggaccacaccatgaaggcattcatgggccgcttgacgcggtccgggctgtttttctggttgccgccggccgccgccgcggtggagttgccgccgccgccccccgaagtttgctgcgggcccggcggcttcagctccgtctccatcatgttgtacatggggccggggttctcctccacgtcgccgcaggtcagcaggctgcccctgccctcgccgctgccaaaatgcctcttcatgtgtaaggcgaggtggtccgacctggaaaatgctcggtcgcatttttggcactggaacgggcggtgccccgtgtgtttacggtagtgcctggtcagttcatctgagcgggcgaatttccatccacagccgtcccagtcacagtggtaaggtttctcacctgtgtgggttcgcaggtgtgccttgagatgggaactctttgtgtaggttttgccgcagcccgcgtaatcacaagtgtgggtggcggtccttttccggggccacgatcgtcttcccctctttggcttgggctcctctggcatgcaggaaccgggtggcatgagctcttggtaatggagcggcgggacttgcggctgcatctgatcgggcaggaaggatgggtaattgggccccgggtggggatggaagccgggaggaagcggcagggcagggtgacagtccctgctgctcagcacttcctcaagacccagggtcggggtagtcctgctggggagctgccgccccagggggaagtcgtgtgcagccggccggtggccattgctgagagggggtccagcgcccaagtgggtgcacgaagagaccgcctcctgcttgatcttggggcacgtgcgcggcggcccgccgttgtagggcgccaccaccaccgggtggctgccgtcagggctgcctttgctgacgctgatgaccgacgggctgccgtactcgctgccaggggcgctcagcgacgccttcagcacgaacttgcccatcagcccgccacctggcggctgcggctgctgcggcggaatgtacaccgggtccaattctggccgcaggagctcggccacgaagccgcccgaggggctcacgtcgttgatgtccgccaggttgaagggagccgtcggagggggagcggactccctgccatagaggaggcctccgcccgtgccgcccggcgccacgcccgggtcgttcccggcccggatcggataggtgaagctgcaggtggagggcgcgctggcagggccgctgctcgacggcgacgacgaagaggaggctgacgctgacgaggacacggtggcggccactgactccggaggatgggtcagcgaattggagagaataaagtccaggtccaggagatcgttgaactcctcggtctctctccgaggtaggggcgccaggttgctaccgccgcaagccgcaccggctccgccgctctccaggtctgtggccacggtcgccgccgccaggtcataggggcggccgggaagcactgggggaagtcgcttcatgtgggagagctcctcccgccagcggttattcggggcacctgcttgacgcagtgtcttctcccttcccgccgggccagacgcgaacgtggagaaagatgggagcagcgcgtcgctgacagccatgggccctgggttggactccacgtctcccgccaacttgagaaggtcaaaattcaaagtctgtttcacgccacttccgtttgaatgcatgggagagcccagagtggtgacggagacagggggaaaggcttccccctcagggaaagggaccgaggagtacagtgcagtgaagtgagggctcccatagcctggggtaccaaaatggggccctggggccagaggaaaggacactggtccccctgagaaaggagacccagcagcctcaaaatcctctcgttgtgcatagtcgctgcttgatcgcttgcccttctggcgccggttacagaaccacactcggaccacatccttctcgagcccaagctgctgggcgatgtggctgatctgctgcagtgtgggtttcgggcactgcaggaacaaattctccaggttgcctctcactcggttctcgatactggttcgctttctctttcgggcctgcacgagggtttctgctttgcatatctcctgaagattttcattgttgtcagcttcctccacccacttctgcagcaagggccgcagcttacacatgttcttgaagctaagctgcagagcctcaaagcggcagatggtcgtttggctgaataccttcccaaatagaacccccagggtgagccccacatcggcctgtgtatatcccagggtgatcctcttctgcttcaggagcttggcaaattgctcgagttctttctgcagagctttgatgtcctgggactcctccgggttttgctccagcttctccttctccagcttcacggcaccaggggtgacggtgcagggctccggggaggccccatcggagttgctctccaccccgactcctgcttcgccctcaggctgagaggtctccaagccgccttggggcactagccccactccaacctggggcccacagtacgccatccccccacagaactcatacggcggggggcatgggggaatcccccacacctcagagcctggcccaacccccggcccgattcctggccctccaggagggccttggaagcttagccaggtccgaggatcaacccagcccggctccggcccccctggcccatcacctccaccacctggagggggcgagaaggcgaaatccgaagccaggtgtcccgccatggtggcgctagcttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgattaattaatcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgagcgattaaacgcgccaactagaggatcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctcgtttatcttatcaattctgtgcctccgggagcagggtagggctgtggatttcttcttcttcctctcgaaagtaggttggctttccctgtgcactagggccctgctgggccttcagcggacatgaggctaccatatggctgatgctctggcagaagtggcacttcttgggctggggtggcagcttgcattccttggcatgatgatctagacctccacagttgtagcacctgtctccttttgatctgcgcttctgcatgctctttccttttggccgcctctcactcccaatacagaatactccaccaggtccggtgacacggatggattccagacccttggctgacttcttaaaggtgaactccactgcctcaccctccttcaagctccggaacccttccatgtgcagcttactctggtgcacaaagacatccactggggggtcgagcgcgaccccggcgcgggcggtcatggacaggaagccgaaccccatgcgcacgttgaaccacttacagatgcccgcaccgtgcagcagctgaggctcgtccgccgcccgggccgcgtcctccggcgcctcctcgggcgcctcttctgccgccttggcgcagccacctgcaaactgctggttggacacggagcccatgggccctgggttggactccacgtctcccgccaacttgagaaggtcaaaattcaaagtctgtttcacgccacttcccacgtcttcaggttgcatgttcatggagtagtttaggaataaatccatggtttgtggagtactaaaatacctagtggtctgctgtattacattaaggccttccccagcagcttccaaggcagcctccaagtcactggcaggagaatttggctggaactgcatgcaggactgcagagattcctctccacagttatagaagggactgttccaggcctgattgttccaggattgggtgcaccaggtctgagtgttccaggagtggttgctccaggactggatgttctgggtctggttgctccaggttgaattgttccaggtctggttgctccacattggaaggttcccagtcgggttcaccaggcatccctggtggtaggaagagtaaaggctggggtaggtaggtgctgaggccttctgcgtcacaccattgctattcttcggccagttgtttttctgccacctcttagatttcattctctggttctggaaccaggtcttcacctgtttgtagctgaggttcaggatgttggagagttcttgcatctgctggaggctgaggtatttctgtctctgaaatctatcattgagtacacacagctgggtggaagagaacacagttctggtcttctgtttcttgaccgggaccttgtcttccttttttgcgacactcttctctgcagaagtgggttgtttgcctttgggactggtggaagaatcagggctgtcctgaataagcagatccatggaggaaggaagaggagagacagtctccgtgtgaggcatctcagcagaagacatttgcaaggatggatagttttcttcaggcccacaaatcacaggcataggtgaagattctttacagtcggatgcttcaaagcaaggcaagctttggggacaagctggatccacactcatggtggcttaatcgcgaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgacccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgc

SEQ ID NO: 3 - DNA, 27879 bp, Artificial Sequence
  Synthetic
ggaactccatatatgggctatgaactaatgaccccgtaattgattactattaataactagtcaataatcaatgtcgagggcgcgccgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgcggccgcgtcgacatttaaatttatgctcttttggcatgcaacatttcaatgagaaggttattatagggcacatccccgttcaggtgcttgtagtagaggtattcttcagcctgcatactgatggcccggatttcgggtagtcgaagaagtagctgtccaaatttctctgtctgctgcgggtagttacacattgtgtagtccagcagggcggcattgacttgttcctggacaccttctaccagctggaagttttcaaggtttttgacatctaaactaaagagcaccaagaatttcagacatacgaactctcgttgatcaaactggagagaacgaagttttgccactaactcctgtgcatgactcatgaggttgttgagggtggctccggcttgtgatgctattatggaatagtccacttgttgcccagtaaccaggaagatggatccttcctttccatgtaccacttgtcggtaaatgtggtcgaggattaagagctcactccagcagttctgaagcagcttcatttggtcatcaaccttaagttctctgaagaagatactactcctggcccactcgacaatggagaagagagtttgatctgccattttgcacataagcccaaaggtgctcagcttttcgtgcttgcttcggttagcctgctcttgctgcaaataggccatgattttagcctggacttgaggctcatctggctcacacttcaaaagttccagtatcagatgtgggatgcttgctggagagctcgtctggtaactatccatatatgaatagcccattatggactcgggtgagctggtataggggtctgggtactcagacttgatggcccggctaggaaagtggccatatgtttggtaaccttgcaggctgccgtgagggggcattgtcatgctaatgggggatgttacaaagggacttctgtcatagtctgtaggaggcaaggcagcatggttcagaggtaggcctttggaggcagagtggatgttttgaattgcagaggaaatggtcaggtcagagggcatagcttggatcacctgagacatggcttctagcttaagtccattggctcggatgagggcttttttctgttgcttcagggccctgtctctcttgtacattggcccaaacttattccttcctccacgcattcggtcggcccttacagcttctagcttcattccaacacttagacatttttgaaaacgacagtaaggacaacgctttctctgtgttttgtcaatttggcagttctggttttctatacatgtgtaccttttattattttggactgttcgcttaaaaaatcccttgcagctttcacaggtgaggagcccataatggtacccagacactttatctccacacacgggacaaagctcttccagatcttcatcataggagtaattcaccattttaaattgagacacaataggtgtaagtccgtgctttaaagactcttgtaaatccccagtatctgaattagaagacatggtggcgatcgctagcttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgattaatcgccgcgccgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggcggatcccatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcggatccgcggccgctcagttagcctcccccatctcccgggcaaacgtgcgcgccaggtcgcatatcgtcggtatggagccgggggtggtgacgtgggtctggaccatcccggaggtaagttgcagcagggcgtcccggcagccggcgggcgattggtcgtaatccaggataaagacgtgcatggaacggaggcgtttggccaagacgtccaaggcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccagtcctcccgccacgacccgccgcactgcagataccgcaccgtattggcaagtagcccgtaaacgcggcgaatcgcagccagcatagccaggtccagccgctcgccggggcgctggcgtttggccaggcggtcgatgtgtctgtcctccggaagggccccaagcacgatgttggtgccgggcaaggtcggcgggatgagggccacgaacgccagcacggcctggggggtcatgctgcccataaggtaccgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccgggcggacttccgtggcttcttgctgccggcgagggcgcaacgccgtacgtcggttgctatggccgcgagaacgcgcagcctggtcgaacgcagacgcgtgttgatggccggggtacgaagccatattaccctgttatccctagaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgacccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagcgctgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttacgccttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcggccgctattacgcacaagagttccgtagctgttcaagtttgtgtttcaactgttctcgtcgtttccgcaacaagtcctcttcagaaatgagcttttgctcctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgaggacctggggctggtgcattttcggttgttgctgatctgtctcaggactctgacactgtccaacttgaccctcttggcagcaggatagtccttccgagtggagggaggcgctgcgtagttgtgctgatgtgtggagacgtggcacctcttgaggaccagtgggctgtgaggaggtttgctgtggcctccagcagaaggtgatccagactctgaccttttgccaggagcctgcctcttttccacagaaacaacatcgatttcttcctcatcttcttgttcctcctcagagtcgctgctggtggtgggcggtgtctcctcatggagcaccaggggctcggggctgccctgcggggaggactccgtcgaggagagcagagaatccgaggacggagagaaggcgctggagtcttgcgaggcgcaggacttgggcgagctgctgtcgttgagagggtaggggaagaccaccgaggggtcgatgcactctgaggcggcggcgctcagatcctgcaggtacaagctggaggtggagcagacgctgtggccgcgggcggggttcgggctgccgctgtctttgcgcgcagcctggtaggaggccagcttctctgagacgagcttggcggcggccgagaagccgctccacatacagtcctggatgatgatgtttttgatgaaggtctcgtcgtccgggtcgcagatgaaactctggttcaccatgtctcctcccagcagctcggtcaccatctccagctggtcggccgtggagaagctcccgccaccgccgtcgttgtctccccgaagggagaagggtgtgaccgcaacgtaggagggcgagcagagcccggagcggcggctaggggacaggggcggggtgggcagcagctcgaatttcttccagatatcctcgctgggcgccgggggctgcagctcgctctgctgctgctgctggtagaagttctcctcctcgtcgcagtagaaatacggctgcaccgagtcgtagtcgaggtcatagttcctgttggtgaagctaacgttgaggggcataggaccggggttactttcaacatcgccagcgagtttcaacaaagcgtagttagtacattgcccactacccatgtgtgagaggggcagtgtgccgttaatggccgtgccgggcaccgggccgctctggtagtgctgggacatgtgaagtctgctgggggcggcgggttccggcacctcggcgccggggagatacatgctgatcatgtcccggaggtccccggcctggcagggcgccctggagtgggaggaagaggtaaccacaggggggctggagctggcctcggacttgaccaccgaacccatggagccaagagccatgccaggggtgccctgctgcgagtaggacatgctgtaggtgggcgagccgttcatgtaggtctgcgagctggtcatggagttgtactgcagggcgctcacgtcgtagcggtgcatgggctgcatctgcgctgcgccgtgcgcattgaggcccgggtgctgcgggtagcccagctggtcctgcatcatgctgtagctgccgttgctccagccgttcatgtgcgcgtaactgtccatgcgctggttcacgcccgcgcccaggccggcgcccaccccgaccccgctcgccatgctattgccgccgggggccagcagcccgccgggcagcgtgtacttatccttcttcatgagcgtcttggttttccgccggggccggtatttataatccgggtgctccttcatgtgcagcgctcgcagccgcttagcctcgtcgatgaacggccgcttctccgtctccgacaaaagtttccactcggcgcccaggcgcttgctgatctccgagttgtgcatcttggggttctcctgggccatcttgcgccgctgcccgcgggaccacaccatgaaggcattcatgggccgcttgacgcggtccgggctgtttttctggttgccgccggccgccgccgcggtggagttgccgccgccgccccccgaagtttgctgcgggcccggcggcttcagctccgtctccatcatgttgtacatggggccggggttctcctccacgtcgccgcaggtcagcaggctgcccctgccctcgccgctgccaaaatgcctcttcatgtgtaaggcgaggtggtccgacctggaaaatgctcggtcgcatttttggcactggaacgggcggtgccccgtgtgtttacggtagtgcctggtcagttcatctgagcgggcgaatttccatccacagccgtcccagtcacagtggtaaggtttctcacctgtgtgggttcgcaggtgtgccttgagatgggaactctttgtgtaggttttgccgcagcccgcgtaatcacaagtgtgggtggcggtccttttccggggccacgatcgtcttcccctctttggcttgggctcctctggcatgcaggaaccgggtggcatgagctcttggtaatggagcggcgggacttgcggctgcatctgatcgggcaggaaggatgggtaattgggccccgggtggggatggaagccgggaggaagcggcagggcagggtgacagtccctgctgctcagcacttcctcaagacccagggtcggggtagtcctgctggggagctgccgccccagggggaagtcgtgtgcagccggccggtggccattgctgagagggggtccagcgcccaagtgggtgcacgaagagaccgcctcctgcttgatcttggggcacgtgcgcggcggcccgccgttgtagggcgccaccaccaccgggtggctgccgtcagggctgcctttgctgacgctgatgaccgacgggctgccgtactcgctgccaggggcgctcagcgacgccttcagcacgaacttgcccatcagcccgccacctggcggctgcggctgctgcggcggaatgtacaccgggtccaattctggccgcaggagctcggccacgaagccgcccgaggggctcacgtcgttgatgtccgccaggttgaagggagccgtcggagggggagcggactccctgccatagaggaggcctccgcccgtgccgcccggcgccacgcccgggtcgttcccggcccggatcggataggtgaagctgcaggtggagggcgcgctggcagggccgctgctcgacggcgacgacgaagaggaggctgacgctgacgaggacacggtggcggccactgactccggaggatgggtcagcgaattggagagaataaagtccaggtccaggagatcgttgaactcctcggtctctctccgaggtaggggcgccaggttgctaccgccgcaagccgcaccggctccgccgctctccaggtctgtggccacggtcgccgccgccaggtcataggggcggccgggaagcactgggggaagtcgcttcatgtgggagagctcctcccgccagcggttattcggggcacctgcttgacgcagtgtcttctcccttcccgccgggccagacgcgaacgtggagaaagatgggagcagcgcgtcgctgacagccatgggccctgggttggactccacgtctcccgccaacttgagaaggtcaaaattcaaagtctgtttcacgccacttccgtttgaatgcatgggagagcccagagtggtgacggagacagggggaaaggcttccccctcagggaaagggaccgaggagtacagtgcagtgaagtgagggctcccatagcctggggtaccaaaatggggccctggggccagaggaaaggacactggtccccctgagaaaggagacccagcagcctcaaaatcctctcgttgtgcatagtcgctgcttgatcgcttgcccttctggcgccggttacagaaccacactcggaccacatccttctcgagcccaagctgctgggcgatgtggctgatctgctgcagtgtgggtttcgggcactgcaggaacaaattctccaggttgcctctcactcggttctcgatactggttcgctttctctttcgggcctgcacgagggtttctgctttgcatatctcctgaagattttcattgttgtcagcttcctccacccacttctgcagcaagggccgcagcttacacatgttcttgaagctaagctgcagagcctcaaagcggcagatggtcgtttggctgaataccttcccaaatagaacccccagggtgagccccacatcggcctgtgtatatcccagggtgatcctcttctgcttcaggagcttggcaaattgctcgagttctttctgcagagctttgatgtcctgggactcctccgggttttgctccagcttctccttctccagcttcacggcaccaggggtgacggtgcagggctccggggaggccccatcggagttgctctccaccccgactcctgcttcgccctcaggctgagaggtctccaagccgccttggggcactagccccactccaacctggggcccacagtacgccatccccccacagaactcatacggcggggggcatgggggaatcccccacacctcagagcctggcccaacccccggcccgattcctggccctccaggagggccttggaagcttagccaggtccgaggatcaacccagcccggctccggcccccctggcccatcacctccaccacctggagggggcgagaaggcgaaatccgaagccaggtgtcccgccatggtggcgctagcttcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgattaattaatagggataacagggtaatttaaatagatctactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagtcgacgagcggccgcagctctaaatactctgaagtccaaagaagttgtatgttgggtgggctcccttcaactttaacatggaagtgctttctgtgactttaaaagtaagtgcttccatgttttagtaggagtgaatccaatttacttctccaaaatagaacacgctaacctcatttgaagggatcccctttgctttaacatgggggtacctgctgtgtgaaacaaaagtaagtgcttccatgtttcagtggaggtgtctccaagccagcacaccttttgttacaaaatttttttgttattgtgttttaaggttactaagcttgttacaggttaaaggattctaactttttccaagactgggctccccaccacttaaacgtggatgtacttgctttgaaactaaagaagtaagtgcttccatgttttggtgatggtaagtcttccttttacatttttattatttttttagaaaataactttattgtattgaccgcagctcatatatttaagctttattttgtatttttacatctgttaaggggccccctctactttaacatggaggcacttgctgtgacatgacaaaaataagtgcttccatgtttgagtgtggtggttcctacctaatcagcaattgcgttaacgcccacactgtgtgcagttcttggctacaggccattactgttgctaatatgcaactctgttgaatataaattggaattgcactttagcaatggtgatggattgttaagccaatgacagaatttaaaccacagacttactttgatagcactcttaatggtataacttcttctcccaggcgcgtctagatcataatcagccataccacattgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagacgtctttaattaatcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgagcgattaaacgcgccaactagaggatcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctcgtttatcttatcaattctgtgcctccgggagcagggtagggctgtggatttcttcttcttcctctcgaaagtaggttggctttccctgtgcactagggccctgctgggccttcagcggacatgaggctaccatatggctgatgctctggcagaagtggcacttcttgggctggggtggcagcttgcattccttggcatgatgatctagacctccacagttgtagcacctgtctccttttgatctgcgcttctgcatgctctttccttttggccgcctctcactcccaatacagaatactccaccaggtccggtgacacggatggattccagacccttggctgacttcttaaaggtgaactccactgcctcaccctccttcaagctccggaacccttccatgtgcagcttactctggtgcacaaagacatccactggggggtcgagcgcgaccccggcgcgggcggtcatggacaggaagccgaaccccatgcgcacgttgaaccacttacagatgcccgcaccgtgcagcagctgaggctcgtccgccgcccgggccgcgtcctccggcgcctcctcgggcgcctcttctgccgccttggcgcagccacctgcaaactgctggttggacacggagcccatgggccctgggttggactccacgtctcccgccaacttgagaaggtcaaaattcaaagtctgtttcacgccacttcccacgtcttcaggttgcatgttcatggagtagtttaggaataaatccatggtttgtggagtactaaaatacctagtggtctgctgtattacattaaggccttccccagcagcttccaaggcagcctccaagtcactggcaggagaatttggctggaactgcatgcaggactgcagagattcctctccacagttatagaagggactgttccaggcctgattgttccaggattgggtgcaccaggtctgagtgttccaggagtggttgctccaggactggatgttctgggtctggttgctccaggttgaattgttccaggtctggttgctccacattggaaggttcccagtcgggttcaccaggcatccctggtggtaggaagagtaaaggctggggtaggtaggtgctgaggccttctgcgtcacaccattgctattcttcggccagttgtttttctgccacctcttagatttcattctctggttctggaaccaggtcttcacctgtttgtagctgaggttcaggatgttggagagttcttgcatctgctggaggctgaggtatttctgtctctgaaatctatcattgagtacacacagctgggtggaagagaacacagttctggtcttctgtttcttgaccgggaccttgtcttccttttttgcgacactcttctctgcagaagtgggttgtttgcctttgggactggtggaagaatcagggctgtcctgaataagcagatccatggaggaaggaagaggagagacagtctccgtgtgaggcatctcagcagaagacatttgcaaggatggatagttttcttcaggcccacaaatcacaggcataggtgaagattctttacagtcggatgcttcaaagcaaggcaagctttggggacaagctggatccacactcatggtggcttaatcgcgaattctttgccaaaatgatgagacagcacaataaccagcacgttgcccaggagctgtaggaaaaagaagaaggcatgaacatggttagcagaggctctagagccgccggtcacacgccagaagccgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgacccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgc

SEQ ID NO: 4 - DNA, 23 bp, Artificial Sequence
  Synthetic
atcagggccaagacatagagatg

SEQ ID NO: 5 - DNA, 20 bp, Artificial Sequence
  Synthetic
gccaatgcaacttggacgtt

SEQ ID NO: 6 - DNA, 23 bp, Artificial Sequence
  Synthetic
tgttcagagccaggaaatccata

SEQ ID NO: 7 - DNA, 26 bp, Artificial Sequence
  Synthetic
cataggtgtctctttctttggtgtgt

SEQ ID NO: 8 - DNA, 24 bp, Artificial Sequence
  Synthetic
atgatggatactttctcggcagga

SEQ ID NO: 9 - DNA, 24 bp, Artificial Sequence
  Synthetic
tgccacgttgtgagttggatagtt

SEQ ID NO: 10 - DNA, 24 bp, Artificial Sequence
  Synthetic
ggacaggtcggtcttgacaaaaag

SEQ ID NO: 11 - DNA, 24 bp, Artificial Sequence
  Synthetic
tctgttgaatgtcgtgaaggaagc

SEQ ID NO: 12 - DNA, 24 bp, Artificial Sequence
  Synthetic
aatccaggataaagacgtgcatgg

SEQ ID NO: 13 - DNA, 24 bp, Artificial Sequence
  Synthetic
gacaatcgcgaacatctacaccac

SEQ ID NO: 14 - DNA, 24 bp, Artificial Sequence
  Synthetic
ataccgcaccgtattggcaagtag

SEQ ID NO: 15 - DNA, 24 bp, Artificial Sequence
  Synthetic
acgtacccgagccgatgacttact

SEQ ID NO: 16 - DNA, 24 bp, Artificial Sequence
  Synthetic
gaagttctcctcctcgtcgcagta

SEQ ID NO: 17 - DNA, 24 bp, Artificial Sequence
  Synthetic
cctgcagtacaactccatgaccag

SEQ ID NO: 18 - DNA, 24 bp, Artificial Sequence
  Synthetic
gtcgcagatgaaactctggttcac

SEQ ID NO: 19 - DNA, 24 bp, Artificial Sequence
  Synthetic
tgtggttacctcttcctcccactc

SEQ ID NO: 20 - DNA, 24 bp, Artificial Sequence
  Synthetic
ttctccgtctccgacaaaagtttc

SEQ ID NO: 21 - DNA, 24 bp, Artificial Sequence
  Synthetic
aagagttcccatctcaaggcacac

SEQ ID NO: 22 - DNA, 24 bp, Artificial Sequence
  Synthetic
ccttcttcatgagcgtcttggttt

SEQ ID NO: 23 - DNA, 24 bp, Artificial Sequence
  Synthetic
tgaactgaccaggcactaccgtaa

SEQ ID NO: 24 - DNA, 24 bp, Artificial Sequence
  Synthetic
gatcgttgaactcctcggtctctc

SEQ ID NO: 25 - DNA, 24 bp, Artificial Sequence
  Synthetic
atgtggtccgagtgtggttctgta

SEQ ID NO: 26 - DNA, 24 bp, Artificial Sequence
  Synthetic
gggtcagcgaattggagagaataa

SEQ ID NO: 27 - DNA, 24 bp, Artificial Sequence
  Synthetic
gatcaagcagcgactatgcacaac

SEQ ID NO: 28 - DNA, 24 bp, Artificial Sequence
  Synthetic
atggctatgggcaacacataatcc

SEQ ID NO: 29 - DNA, 24 bp, Artificial Sequence
  Synthetic
ctctcagcgacctcgtgaatatga

SEQ ID NO: 30 - DNA, 24 bp, Artificial Sequence
  Synthetic
cacaaaccccttgggcaataaata

SEQ ID NO: 31 - DNA, 24 bp, Artificial Sequence
  Synthetic
ccattagtggttttgtgggcaagt

SEQ ID NO: 32 - DNA, 24 bp, Artificial Sequence
  Synthetic
cctcatctccatcacctccttcat

SEQ ID NO: 33 - DNA, 24 bp, Artificial Sequence
  Synthetic
tccaacccgaaatttgagaacatt

SEQ ID NO: 34 - DNA, 24 bp, Artificial Sequence
  Synthetic
ggaaaccagggaggcaaatctact

SEQ ID NO: 35 - DNA, 24 bp, Artificial Sequence
  Synthetic
tcacgtagaaaggactaccgacga

SEQ ID NO: 36 - DNA, 24 bp, Artificial Sequence
  Synthetic
aacccttccatgtgcagcttactc

SEQ ID NO: 37 - DNA, 23 bp, Artificial Sequence
  Synthetic
ctgctggggaaggccttaatgta

SEQ ID NO: 38 - DNA, 24 bp, Artificial Sequence
  Synthetic
cgcctctcactcccaatacagaat

SEQ ID NO: 39 - DNA, 24 bp, Artificial Sequence
  Synthetic
gaagtggcgtgaaacagactttga

SEQ ID NO: 40 - DNA, 24 bp, Artificial Sequence
  Synthetic
tgtcaatttggcagttctggtttt

SEQ ID NO: 41 - DNA, 24 bp, Artificial Sequence
  Synthetic
aggggttttatgcgatggagtttc

SEQ ID NO: 42 - DNA, 24 bp, Artificial Sequence
  Synthetic
ggacaacgctttctctgtgttttg

SEQ ID NO: 43 - DNA, 24 bp, Artificial Sequence
  Synthetic
ccagcttggcacttgatgtaattc

SEQ ID NO: 44 - DNA, 21 bp, Artificial Sequence
  Synthetic
gcactagccccactccaacct

SEQ ID NO: 45 - DNA, 24 bp, Artificial Sequence
  Synthetic
aggggttttatgcgatggagtttc

SEQ ID NO: 46 - DNA, 21 bp, Artificial Sequence
  Synthetic
gagttgctctccaccccgact

SEQ ID NO: 47 - DNA, 24 bp, Artificial Sequence
  Synthetic
ccagcttggcacttgatgtaattc

SEQ ID NO: 48 - DNA, 25 bp, Artificial Sequence
  Synthetic
atggtgatggattgttaagccaatg

SEQ ID NO: 49 - DNA, 25 bp, Artificial Sequence
  Synthetic
tggacaaaccacaactagaatgcag

SEQ ID NO: 50 - DNA, 24 bp, Artificial Sequence
  Synthetic
aagctgaccctgaagttcatctgc

SEQ ID NO: 51 - DNA, 25 bp, Artificial Sequence
  Synthetic
cttctgcttgtcggccatgatatag

(19) United States
(12) Patent Application Publication
(10) Pub. No.: US 2017/0327796 A1
(43) Pub. Date: Nov. 16, 2017

(54) INDUCED PLURIPOTENT STEM CELL AND METHOD FOR PRODUCING THE SAME

(71) Applicant: UNIVERSITY OF UTAH RESEARCH FOUNDATION, Salt Lake City, UT (US)

(72) Inventors: Sen Wu, Salt Lake City, UT (US); YuanYuan Wu, Salt Lake City, UT (US); Mario Capecchi, Salt Lake City, UT (US)

(21) Appl. No.: 15/531,145

(22) PCT Filed: Jul. 1, 2015
(86) PCT No.: PCT/US2015/038893

§ 371 Date: May 26, 2017

Related U.S. Application Data
(60) Provisional application No. 62/093,811, filed on Dec. 18, 2014.

(51) Int. Cl. C12N 5/074; C12N 15/85

(52) U.S. Cl. CPC C12N 5/0696; C12N 15/85; C12N 2506/02; C12N 2501/727; C12N 2501/65; C12N 2501/608; C12N 2501/606; C12N 2501/604; C12N 2501/603; C12N 2501/602; C12N 2501/60; C12N 2500/90; C12N 2510/00; C12N 2501/605

(57) ABSTRACT

The disclosure provides an episome comprising OCT4, KLF4, SOX2, cMYC, NANOG, LIN28, and NRSA2. Also disclosed is a method for producing an induced pluripotent stem (iPS) cell. The method comprises introducing an episome into a cell, wherein the episome comprises OCT4, KLF4, SOX2, cMYC, NANOG, LIN28, and NRSA2, and growing the cell under conditions to select for the presence of the episome. The method also comprises selecting a primary clone and growing the primary clone in a medium comprising a MEK inhibitor and a GSK3b inhibitor.

### RELATED APPLICATION DATA

This application claims priority to U.S. Provisional Application No. 62/093,811 filed Dec. 18, 2014, the entire contents of which are incorporated herein by referenced.

### TECHNICAL FIELD

The present disclosure provides episomes for producing induced pluripotent stem (iPS) cells, methods for producing iPS cells, and methods for treating disease using the iPS cells.

### BACKGROUND

iPS cells are adult cells that have been genetically reprogrammed to an embryonic stem cell-like state. iPS cells propagate indefinitely and give rise to other cell types. Because iPS cells are derived from an adult cell, the resulting pluripotent stem cell line can be matched to the subject from which the adult cell was obtained. Accordingly, iPS cells may be used in cell-based therapies and regenerative medicine.

iPS cells may be generated by methods that employ viral vectors to randomly integrate genes into somatic cells. Such viral methods are associated with insertional mutagenesis and may thus result in the induction of cancer in the recipient as well as increased morbidity and mortality. Other approaches to deriving iPS cells have used non-viral means such as plasmids, the piggyback (“PB”) transposon, non-integrating episomes, protein transduction, transfection of mRNA and microRNAs, and small molecules inhibitors. A need still exists, however, for a way to produce transgene-free, germ line competent iPS cells (i.e., iPS cells that contribute to a functional germ line in reproductively competent animals).

### SUMMARY

The present disclosure provides episomes comprising OCT4, KLF4, SOX2, cMYC, NANOG, LIN28, and NR5A2.

The present disclosure also provides methods for producing induced pluripotent stem (iPS) cells. These methods may comprise introducing an episome into a cell, wherein the episome comprises OCT4, KLF4, SOX2, cMYC, NANOG, LIN28, and NR5A2, and growing the cell under conditions to select for the presence of the episome. The method may also comprise selecting a primary clone and growing the primary clone in a medium comprising a MEK inhibitor and a GSK3b inhibitor.

### BRIEF DESCRIPTION OF THE DRAWINGS

FIG. 1 shows the generation of transgene-free iPS cells using non-integrating episomal vectors. (A) Map of non-integrating vector pMaster1. (B) Map of non-integrating vector pMaster3. (C) Map of non-integrating vector pMaster12. The pMaster1 episome has six reprogramming genes: OCT4, KLF4, SOX2, and cMYC (collectively denoted as “OKSM”), NANOG, and LIN28. The pMaster3 episome has seven reprogramming genes: OKSM (OCT4, KLF4, SOX2, and cMYC), NANOG, LIN28, and NR5A2). And the pMaster12 episome has eight reprogramming genes, which include OKSM (OCT4, KLF4, SOX2, and cMYC), NANOG, LIN28, NR5A2, and the miR302/367 cluster. Each vector has positive and negative selection genes driven by the CAG promoter and Epstein-Barr virus derived EBNA-1 and ori-P loci for stable episomal maintenance. (D) Outline of exemplary steps used to reprogram and subsequently select for iPS cells that have lost the pMaster episomes. The iPS cell lines were derived in serum medium, and picked and expanded colonies were subjected to FIAU negative selection in the 2i medium. The obtained secondary clones were then grown in either serum or 2i medium for more than 3 passages before use, depending on the subsequent analysis. The 2i transcriptome and serum transcriptome of the same line were not fixed, and may be interchangeable, upon growth in the relevant medium for 2-3 passages. (E) Primary clones had typical ES cell morphology and showed GFP expression from an endogenous Oct4-GFP transgene. (F) Following negative selection using FIAU, some clones differentiated and some showed undifferentiated ES cell-like morphology. Established transgene-free, FIAU-resistant clones showed morphology typical of mouse ES cell colonies and homogeneous GFP expression from the Oct4-GFP transgene signifying endogenous Oct4 gene expression.

FIG. 2 shows in vitro characterization of pMaster12-derived transgene-free iPS cells. (A) PCR confirmation of removal of pMaster12 vector in the iPSZX11-18-2 cell line. In clone iPSZX11-18-2, pMaster12 sequences were not detected by PCR using primer pairs 1-21 (FIG. 5 and Table 3) covering the entire pMaster12 vector, while the endogenous Oct4-GFP transgene was detected. Purified pMaster12 DNA was used as control. (B) Immunostaining of transgene-free iPSZX11-18-2 cells demonstrated that the endogenous pluripotency genes (e.g. OCT4, SOX2, NANOG, and SSEA1) were active after the loss of pMaster12 vector. FIG. 2(C) shows the normal female karyotype of iPSZX11-18-2 cells.

FIG. 3 shows germ line transmission of pMaster-derived iPS cell lines. (A) Transgene-free iPS cell line iPSZX11-18-2 was generated with pMaster12, and blastocyst injection with those cells produced 11 female chimeras (one example shown in left panel), two of which passed iPS-derived cells to their progeny at high frequencies (44% by coat color, 33% by Oct4-GFP). (B) The iPS466F38 cell line was generated using pMaster3 and 129Sv/BL6 Oct4-GFP MEFs. Blastocyst injection produced ten male chimeras (one example shown in left panel). Nine chimeras were mated, and seven produced iPS-derived progeny at very high frequencies (45%). Since these iPS cells only had one copy of the agouti gene and one copy of the Oct4-GFP gene, the maximum expected germ line transmission frequency detected via either marker is 50%.

FIG. 4 shows differential gene expression in iPS cells grown in 2i medium (containing small molecule inhibitors of the MEK and GSK3b pathways) vs. serum medium and functional annotation through GO terms and KEGG pathways. (A) Hierarchical clustering of seven iPS cell lines (pMaster1: iPS322-38s, iPS322-40t, iPS344F28 and iPS344F30; pMaster3: iPS466F38 and iPS466F46), two ES cell lines (R1 and G4), and two MEF lines (wt 129Sv and 129Sv/BL6 Fl Oct4-GFP), cultured in 2i medium or serum medium. All of the pMaster1 cell lines retained the six transgenes (OKSM, NANOG and LIN28). The pMaster3 line iPS466F38 retained NR5A2, and line iPS466F46 was transgene-free. All of the lines analyzed except iPS466F46 had normal karyotype. Six of those pMaster1 or 3 cells (Table 2) grown in 2i and used for blastocyst injection gave germ line transmission. All of the iPS and ES cell lines were male lines. Each iPS or ES cell line was grown in ES cell media and 2i media, for more than 3 passages before use. Hierarchical clustering was performed using 6144 significantly different genes. (B) Differentially expressed genes in 2i medium vs serum medium (>2-fold). Pair-wise comparison revealed that 687 genes expressed more than 2-fold higher in 2i medium, while 1295 genes expressed more than 2-fold higher in serum medium. (C) Expression of genes associated with development of three germ layers. Most of these typical marker genes related to various germ layers activated in serum medium compared to 2i medium. (D) Genes upregulated in 2i medium were enriched for GO terms associated with immune response and metabolic processes. (E) Genes upregulated in serum medium were enriched for GO terms associated with cell differentiation, tissue and organ development. (F) Genes upregulated in 2i medium were enriched for KEGG terms linked to glycosphingolipid biosynthesis, metabolism of xenobiotics by cytochrome P450, complement and coagulation cascades, and glutathione metabolism. (G) Genes upregulated in serum medium were enriched for KEGG terms linked to VEGF signaling pathway, axon guidance, hematopoietic cell lineage, etc.

FIG. 5 shows PCR validation of episome removal in pMaster-derived iPS cells. (A) PCR showed partial loss of episomal sequences in pMaster1 iPS mice. Lane 1: neo-IRES, WS1384/1385; Lane 2: neo-IRES, WS1386/1387; Lane 3: tk, ws 1388/1389; Lane 4: tk, WS 1390/1391; Lane 5: MYC-SOX2, WS1392/1393; Lane 6: MYC-SOX2, WS1394/1395; Lane 7: SOX2-KLF4, WS1396/1397; Lane 8: SOX2-KLF4, WS1398/1399; Lane 9: KLF4-OCT4, WS1400/1401; Lane 10: KLF4-OCT4, WS1402/1403; Lane 11: oriP, WS1404/14; Lane 12: oriP, WS1406/1407; Lane 13: EBNA 1, WS1408/1409; Lane 14: EBNA1 , WS1410/1411; Lane 15: Amp-pUC, WS1412/1413; Lane 16: Amp-pUC, WS1414/1415; Lane 17: NANOG-LIN28, WS1416/1417; and Lane 18: NANOG-LIN28, WS1418/1419. (B) PCR showed partial loss of episomal sequences in pMaster3 iPS mice, with only NR5A2 retained. Lane 1: neo-IRES, WS1384/1385; Lane 2: neo-IRES, WS1386/1387; Lane 3: tk, WS1388/1389; Lane 4: tk, WS1390/1391; Lane 5: MYC-SOX2, WS1392/1393; Lane 6: MYC-SOX2, WS1394/1395; Lane 7: SOX2-KLF4, WS1396/1397; Lane 8: SOX2-KLF4, WS1398/1399; Lane 9: KLF4-OCT4, WS1400/1401; Lane 10: KLF4-OCT4, WS1402/1403; Lane 11: oriP, WS1404/1405; Lane 12: oriP, WS1406/1407; Lane 13: EBNA1, WS1408/1409; Lane 14: EBNA, WS1410/1411; Lane 15: NANOG-LIN28, WS1416/1417; Lane 16: NANOG-LIN28, WS1418/1419; Lane 17: NR5A2, WS1555/1552; Lane 18: NR5A2, WS1556/1554; Lane 19: EF1a-OCT4, WS1551/1552; and Lane 20: EF1a-OCT4, WS1553/1554. (C) PCR showed complete removal of transgenes in pMaster12 iPS mice. Lane 1: neo-IRES, WS1384/1385; Lane 2: neo-IRES, WS1386/1387; Lane 3: tk, WS1388/1389; Lane 4: tk, WS1390/1391; Lane 5: MYC-SOX2, W$1392/1393; Lane 6: MYC-SOX2, WS1394/1395; Lane 7: SOX2-KLF4, WS1396/1397; Lane 8: SOX2-KLF4, WS1398/1399; Lane 9: KLF4-OCT4, WS1400/1401; Lane 10: KLF4-OCT4 , WS1402/1403; Lane 11: oriP, WS1404/1405; Lane 12: oriP, WS1406/1407; Lane 13: EBNA1 , WS1408/1409; Lane 14: EBNA1 , WS1410/1411; Lane 15: NANOG-LIN28, WS1416/1417; Lane 16: NANOG-LIN28, WS1418/1419; Lane 17: NR5A2, WS1555/1552; Lane 18: NR5A2, WS1556/1554; Lane 19: EF1a-OCT4, WS1551/1552; Lane 20: EF1a-OCT4, WS1553/1554; and Lane 21: Mir302/367, WS1645/1646.

FIG. 6 shows karyotypes of pMaster vectors-derived iPS cells. (A) iPS344F28 (pMaster1) cells had a normal male karyotype. (B) iPS344F30 (pMaster1) cells had a normal male karyotype. (C) iPS466F38 (pMaster3) cells had a normal male karyotype. (D) iPS466F46 (pMaster3) cells had an abnormal male karyotype, with duplication in the long arm of one copy of chromosome 5. (E) iPSZX11-18-1 (pMaster12) cells had a normal female karyotype.

FIG. 7 shows germ line transmission of additional pMaster vector-derived iPS cells. (A) iPS cell line iPS322-38s was generated from 129Sv mouse embryonic fibroblasts (MEFs) using pMaster1. Transgenes were only partially removed. Blastocyst injection produced a single chimera that gave germ line progeny. (B) iPS cell line iPS344F28 was generated from 129Sv/BL6 Oct4-GFP MEFs using pMaster1. Transgenes were only partially removed. Blastocyst injection produced two male chimeras. Chimera #7827 produced germ line progeny. (C) iPS cell line iPS344F30 was generated from 129Sv/BL6 Oct4-GFP MEFs using pMaster1. Transgenes were only partially removed. Blastocyst injection produced four male chimeras, three of which gave germ line progeny. (D) Transgene-free iPS cell line iPS466F46 was generated from 129Sv/BL6 Oct4-GFP MEFs using pMaster3 . Although this line had an abnormal karyotype (FIG. 6(D)), blastocyst injection produced 6 male chimeras, 2 of which have given germ line progeny. (E) Transgene-free iPS cell line iPSZX11-18-2 was generated from 129Sv/BL6 Oct4-GFP MEFs using pMaster12. Blastocyst injection produced 11 female chimeras.

FIG. 8 shows functional annotation of higher differentially expressed genes between pMaster iPS cells and G4 ES cells by analyses through GO terms and KEGG pathways. Upregulated genes in pMaster1 iPS cells vs. G4 ES cells were enriched for GO and KEGG terms associated with cell differentiation and organ development, and in particular, neural development. Upregulated genes in pMaster3 iPS cells vs. G4 ES cells were enriched for GO and KEGG terms associated with sensory organ development, cell surface receptor linker signal transduction. Upregulated genes in pMaster12 iPS cells vs. G4 ES cells were enriched for much fewer GO and KEGG terms associated cell differentiation and development. (A) Upregulated genes in pMaster1 vs. G4 ES cells were enriched for KEGG terms. (B) Upregulated genes in pMaster3 vs. G4 ES cells were enriched for KEGG terms. (C) Upregulated genes in pMaster12 vs. G4 ES cells were enriched for KEGG terms. (D) Upregulated genes in pMaster1 vs. G4 ES cells were enriched for GO terms. (E) Upregulated genes in pMaster3 vs. G4 ES cells were enriched for GO terms. (F) Upregulated genes in pMaster12 vs. G4 ES cells were enriched for GO terms.

FIG. 9 shows functional annotation of lower differentially expressed genes between pMaster iPS cells and G4 ES cells by analyses through GO terms and KEGG pathways. Downregulated genes in pMaster1 iPS cells vs. G4 ES cells were enriched for GO and KEGG terms associated with immune function, cell adhesion molecules, signal transduction, and metabolic processes. Downregulated genes in pMaster3 iPS cells vs. G4 ES cells were enriched for GO and KEGG terms associated with immune function, cell adhesion molecules, and proteolysis. Downregulated genes in pMaster12 iPS cells vs. G4 ES cells were enriched for GO and KEGG terms associated with ion transport. (A)

Downregulated genes in pMaster1 vs. G4 ES cells were enriched for KEGG terms. (B)

Downregulated genes in pMaster3 vs. G4 ES cells were enriched for KEGG terms. (C)

Downregulated genes in pMaster12 vs. G4 ES cells were enriched for KEGG terms. (D)

Downregulated genes in pMaster1 vs. G4 ES cells were enriched for GO terms. (E)

Downregulated genes in pMaster3 vs. G4 ES cells were enriched for GO terms. (F)

Downregulated genes in pMaster12 vs. G4 ES cells were enriched for GO terms vs. G4 ES cells were enriched for GO terms.

FIGS. 10A, 10B, and 10C show differentially expressed genes in group 1 (pMaster1 iPS cell lines) and group 2 (pMaster3 iPS cell lines+2 ES cell lines).

FIG. 11 shows pMaster vectors with species specific miR302/367 cluster. It appeared that the additional microRNA 302/367 gene cluster on the pMaster12 vector was beneficial for obtaining high-quality iPS cells. We have further constructed similar vectors each with a species-specific miR302/367 gene cluster for (A) pig, (B) cow, (C) sheep, (D) bat, and (E) rat.

### DETAILED DESCRIPTION

The disclosure provides episomes for producing induced pluripotent stem (iPS) cells. The episomes may promote the formation of iPS cells that are transgene-free and germ line competent. As discussed in more detail below, episomes comprising the genes OCT4, KLF4, SOX2, cMYC, NANOG, LIN28, NR5A2, and microRNA 302/367 gene cluster, and the selection markers neomycin resistance and HSV-tk, may promote the formation of iPS cells that are transgene-free and germ line competent.

The disclosure also provides methods for producing iPS cells. The resulting iPS cells may have the advantageous properties of being transgene-free and germ line competent. These methods may generate iPS cells with an efficiency of at least about 0.2%.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Germ line competent,” “germ line competence,” or “germ line competency” as used herein refers to a cell or cell line that contributes to germ cell formation and transmits a targeted gene(s) to progeny. In some embodiments, germ line competence of the cell or cell line may be determined by breeding of a chimeric subject that harbors a mutation and a wild-type subject, and genotyping the progeny for the presence of the mutation (i.e., the targeted gene).

The term “subject” as used herein means a mammal, a bird, or a reptile. The subject may be a mouse, cow, horse, dog, cat, or a primate. The subject may be a human.

“Treat,” “treating,” or “treatment” as used herein interchangeably refers to reversing, alleviating, or inhibiting the progress of a disease, or one or more symptoms of such disease, to which such term applies. Depending on the condition of the subject, the term also refers to preventing a disease, and includes preventing the onset of a disease, or preventing symptoms associated with a disease. A treatment may be performed in an acute or chronic way. The term also refers to reducing the severity of a disease or symptoms associated with such disease prior to affliction with the disease. “Preventing” also refers to preventing the recurrence of a disease or one or more symptoms associated with such disease. “Treatment” and “therapeutically” refer to the act of treating as “treating” is defined above.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. METHODS FOR PRODUCING INDUCED PLURIPOTENT STEM (IPS) CELLS

Provided herein are methods for producing induced pluripotent stem (iPS) cells. These methods provide iPS cells that are transgene-free and exhibit germ line competence. The methods may be used to generate iPS cells at an efficiency of at least about 0.05%, 0.06%, 0.07%, 0.08%, 0.09%, 0.10%, 0.20%, 0.30%, 0.40%, 0.50%, 0.60%, 0.70%, 0.80%, 0.90%, or 1.00%. The method may generate the iPS cell at an efficiency of at least about 0.20%. Efficiency may be calculated by dividing the number of obtained iPS colonies by the number of transfected cells.

The methods may include introducing an episome into a cell and growing the cell under conditions to select for the presence of the episome, thereby identifying a primary clone. The methods may also include selecting for the absence of the episome, thereby identifying a secondary clone. The methods may further include verifying this secondary clone is transgene-free. Such a transgene-free clone may be grown in a medium to yield the iPS cell.

a. Episome

The methods include introducing the episome into the cell. The cell is described below in more detail. The episome may contain one or more genes. The one or more genes may reprogram the cell into the iPS cell.

The one or more genes may be OCT4, KLF4, SOX2, cMYC, NANOG, LIN28, NR5A2, or microRNA 302/367 cluster, or any combination thereof. The one or more genes may be OCT4, KLF4, SOX2, cMYC, NANOG, and LIN28. In other embodiments, the one or more genes may be OCT4, KLF4, SOX2, cMYC, NANOG, LIN28, and NR5A2. In still other embodiments, the one or more genes may be OCT4, KLF4, SOX2, cMYC, NANOG, LIN28, NR5A2, and microRNA 302/367 cluster.

The episome may contain a polycistronic locus expressed from a single promoter. The polycistronic locus may include a sequence encoding a 2A peptide. This sequence encoding the 2A peptide may be positioned between adjacent members of the polycistronic locus. Members of the polycistronic locus may include at least two of the genes OCT4, KLF4, SOX2, cMYC, NANOG, LIN28, NR5A2, and microRNA 302/367 gene cluster. Members of the polycistronic locus may include at least two of the genes OCT4, KLF4, SOX2, cMYC, NANOG, LIN28, and NR5A2. Members of the polycistronic locus may include the genes OCT4, KLF4, SOX2, and cMYC.

The episome may contain one or more selection markers. The one or more selection markers may be a positive selection marker, a negative selection marker, or a combination thereof The positive selection marker may be neomycin resistance. The negative selection maker may be HSV-tk. In some embodiments, the one or more selection markers may be neomycin resistance and HSV-tk.

The episome may further contain EBNA-1/oriP.

SEQ ID NO:1=pMaster1; SEQ ID NO:2=pMaster3; SEQ ID NO:3=pMaster12.

b. Cell

As described above, the episome is introduced into the cell and the cell is then grown under conditions to select for the presence of the episome as described below in more detail. The cell may be a somatic cell. The cell may be, for example, a fibroblast cell, a mesenchymal stem cell, a keratinocyte, a blood cell, a hepatocyte, or a urine cell.

c. Inhibitor Medium

As described in more detail below, the methods may include growing the transgene-free clone in an inhibitor medium. The inhibitor medium improves the pluripotency of the transgene-free cell, which is described below in more detail, as compared to a pluripotency of a transgene-free cell that is not grown in inhibitor medium.

The inhibitor medium may comprise a MEK inhibitor, a GSK3b inhibitor, or a MEK inhibitor and a GSK3b inhibitor. The inhibitor medium may be serum free. In some embodiments, the inhibitor medium may be serum free and include a MEK inhibitor and a GSK3b inhibitor (2i medium).

d. Introduction of the Episome into the Cell

The methods include introducing the episome into the cell. Introduction may include forming a mixture of the cell and the episome. Introduction may also include electroporation of this mixture, thereby resulting in uptake of the episome by the cell. Introduction may also include transfection using methods and reagents familiar to those of ordinary skill in the art.

e. Selection for the Presence of the Episome

After introduction of the episome into the cell, the cell may be grown under conditions that select for the presence of the episome within the cell. This, in turn, allows for the selection of a primary clone.

Selection for the presence of the episome may include incubating the cell in the presence of G418. The selection marker neomycin provides resistance to G418, and thus, if a cell is resistant to G418, then the episome is present within the cell. The primary clone is the cell that is resistant to G418.

f. Selection for the Absence of the Episome

After selection of the primary clone, the primary clone may be grown under conditions that select for the absence of the episome within the cell. This, in turn, allows for the selection of a secondary clone.

Selection for the absence of the episome may include incubating the primary clone in the presence of 1-(2-deoxy-2-fluoro-1-D-arabinofuranosyl)-5-iodouracil (FIAU). FIAU is lethal to a cell that contains the selection marker HSV-tk. Accordingly, resistance to FIAU in a primary clone indicates loss of the episome. The secondary clone is the cell that is resistant to FIAU.

g. Verifying that a Secondary Clone is Transgene-Free

After selection of a secondary clone, it may be verified that the secondary clone is transgene-free. Verification may include, but is not limited to, isolation of DNA from the secondary clone and subjecting the isolated DNA to the polymerase chain reaction (PCR) in the presence of primers specific for the one or more genes present on the episome. The one or more genes contained in the episome are described above. If an amplicon is not generated for the one or more genes, then this result indicates that the secondary clone is a transgene-free clone. However, if an amplicon is generated for the one or more genes, then this result indicates that the secondary clone is not a transgene-free clone. Southern blot analysis could also be used to verify whether a secondary clone is transgene-free.

h. Growing the Transgene-Free Clone in an Inhibitor Medium

After identification of the transgene-free clone, this transgene-free clone may be grown in inhibitor medium, which is described above. The selected inhibitor medium may improve the pluripotency of the transgene-free cell as compared to a pluripotency of a transgene-free cell not grown in inhibitor medium. Pluripotency can be assessed, for example, by examining the percentage of chimerism or frequency of germline transmission obtained with a particular clone. This transgene-free cell, having improved pluripotency because it was grown in inhibitor medium, is the iPS cell.

### 3. METHODS FOR TREATING A DISEASE

Also provided herein are methods for treating a disease in a subject in need thereof. The methods may include producing an iPS cell by the method described above, or providing an iPS cell as described above. The methods also may include administering an iPS cell to the subject. The iPS cell may be produced using somatic cells obtained from the subject. Administration may include, but is not limited to, intravenous delivery, subcutaneous delivery, intramuscular delivery, and implantation.

Implantation may include generating a tissue from the iPS cell and providing the tissue to the subject. The tissue may be, for example, a dermal tissue, a vascular tissue, insulin-producing beta cells, cardiac cells.

The disease may include, but is not limited to, diabetes.

### 4. METHODS FOR USING IPS CELLS

Also provided herein are methods for using iPS cells that include differentiating the iPS cells according to any methods that are currently known or hereinafter devised.

### 5. KITS

Further provided herein are kits that include the episome. The kit may be used in the methods described above. The kit may also include other material(s), which may be desirable from a user standpoint, such as a buffer(s), a diluent(s), a standard(s), and/or other material useful in conducting any step of the method described herein.

The kits preferably may include instructions for carrying out the disclosed methods. Instructions included in the kit may be affixed to packaging material or may be included as a package insert. While instructions may include written materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site which provides instructions.

The present invention has multiple aspects, illustrated by the following non-limiting examples.

### 6. EXAMPLES

### Example 1

### Materials and Methods for Example 2

Plasmid Construction. To construct the pMaster series of vectors, the EF1a promoter-driven OKSM expression cassette was used as well as the CAG promoter-driven positive/negative selection cassette comprising neomycin resistance and HSV-tk. NANOG and LIN28 cDNAs were fused with an intervening F2A sequence by PCR. Components were assembled onto the pCEP4 episomal mammalian expression vector backbone that contains the Epstein-Barr virus replication origin (oriP) and nuclear antigen (encoded by the EBNA1 gene).

Cell Culture and Media. Mouse embryonic fibroblasts (MEFs) from strain 129Sv were isolated from pooled embryonic day 14 (E14) embryos. To isolate MEFs from the Oct4-GFP transgenic mouse line (Jax Mice strain name: B6; CBA-Tg(Pou5f1-EGFP)2Mnn/J; stock number 004654), a homozygous Oct4-GFP male was crossed to 129Sv females, and E14 MEFs from pooled F1 heterozygous embryos were isolated.

ES medium (serum medium) was prepared by supplementing DMEM medium with 15% fetal bovine serum, 500 units/ml Lif, and 0.1 mM 2-mercaptoethanol.

2i medium was made by mixing 500 ml of DMEM/F12 medium (Invitrogen 10565-042), 500 ml of Neurobasal medium (Invitrogen 21103-049), 5 ml of N2 supplement (Invitrogen 17502-048), 10 ml of B27 supplement (Invitrogen 17504-044), 5 ml of 100× Pen-Strep (Invitrogen 15070-063), 2-mercaptoethanol (final 0.1 mM), Lif (Millipore, ESG1107, final 1000 units/ml), PD0325901 (Selleck, final 1 μM), and CHIR99021 (Selleck, final 3 μM).

Reprograming MEFs with pMaster Vectors. For each electroporation experiment, 2×10⁶ MEFs were used, and 3 μg of pMaster DNA was transfected using the Lonza Amaxa Nucleofector, program A-024. Electroporated cells were transferred into pre-warmed ES medium (serum medium) and plated onto 10-cm culture plates containing irradiated feeders. AT 20-24 hours after transfection, G418 selection was applied at 350 μg/ml. G418 selection was ended after five days. Colonies began to appear at about 8 days after electroporation, and clones were picked into 24-well dishes containing ES medium (serum medium) on about day 10-15. When clones grew to around 50-60% confluent in the 24-well dish (after 2-3 days), about 2×10⁴ cells from each clone were seeded on a 60-mm plate with 0.2 μM FIAU in 2i medium. FIAU selection was maintained for 5 days. About 5 days after FIAU selection was stopped, 4 secondary clones from each 60-mm plate were picked into 24-well dishes. When 50-60% confluent, each of these secondary clones was passaged and split into 3 wells in a 12-well dish (one for cryopreservation, one for genomic DNA preparation, and one to test for G418 sensitivity).

Real-Time PCR. To quantify copy number of the pMaster12 plasmid in iPS cells, real time PCR was performed with Roche LightCycler® 480. To generate a standard curve, pMaster12 plasmid was mixed with MEF genomic DNA, at 1, 2, 4, 8, 16, and 32 copies per cell as standards, according to the formula: pMaster12 (ng)/MEF genomic DNA (ng)=pMaster12 size (27879 bp)/mouse genome size (2.726×10⁹ bp). ACt=Ct for EBNA—Ct for Fabp2 gene, the endogenous gene control. ACt values were plotted against the number of cycles on a logarithmic scale to obtain the standard curve. EBNA primers used were (EBNA-F: ATC AGG GCC AAG ACA TAG AGA TG (SEQ ID NO:4)) and (EBNA-R: GCC AAT GCA ACT TGG ACG TT (SEQ ID NO:5)), and the product size was 60 bp. Fabp2 primers used were (Fabp2-F TGT TCA GAG CCA GGA AAT CCA TA (SEQ ID NO:6)) and (Fabp2-R CAT AGG TGT CTC TTT CTT TGG TGT GT (SEQ ID NO:7)), and the product size was 110 bp. The copy number of EBNA in each sample was estimated based on the ACt value.

Microarray Analysis. Miroarray analysis was performed using Agilent Mouse Gene Expression Microarray. Total RNA was prepared using the Qiagen RNeasy kit. The Agilent One-Color Quick Amp Labeling Kit was used to generate fluorescently label cRNA for one-color microarray hybridizations. Microarray hybridizations were performed using Agilent SureHyb Hybridization chambers. Microarray slides were scanned in an Agilent Technologies G2505C Microarray Scanner. The normalized data set was loaded into GeneSifter (Geospiza) for analysis.

Karotyping. iPS cells were treated with Colcemid (0.1 μg/ml) (Gibco, cat #15212-012) for 20 to 30 minutes. Metaphase chromosomes were prepared using a standard procedure. Chromosomes were analyzed using standard GTW banding method.

Immunostaining. Cells were fixed in 4% paraformaldehyde in PBS for 30 min at room temperature and standard procedures were used for immunostaining with the following antibodies: polyclonal rabbit anti-Sox2 (Novus, NB110-37235), mouse IgM anti-SSEA1 (DSHB, MC-480), polyclonal rabbit anti-Nanog (Abcam, ab80892), and mouse IgG2b anti-Oct4 (Santa cruz, sc-5279). After staining, cells were washed with PBS and nuclei were counterstained with DAPI.

Chimera Production. Chimera production was done by morula aggregation and blastocyst injection. Laser assisted injection of eight-cell stage embryos was performed. Recipient embryos for morula aggregation were from the CD1 mouse line. Recipient embryos for eight-cell or blastocyst injection were from the C57/BL6 mouse line.

### Example 2

### Results

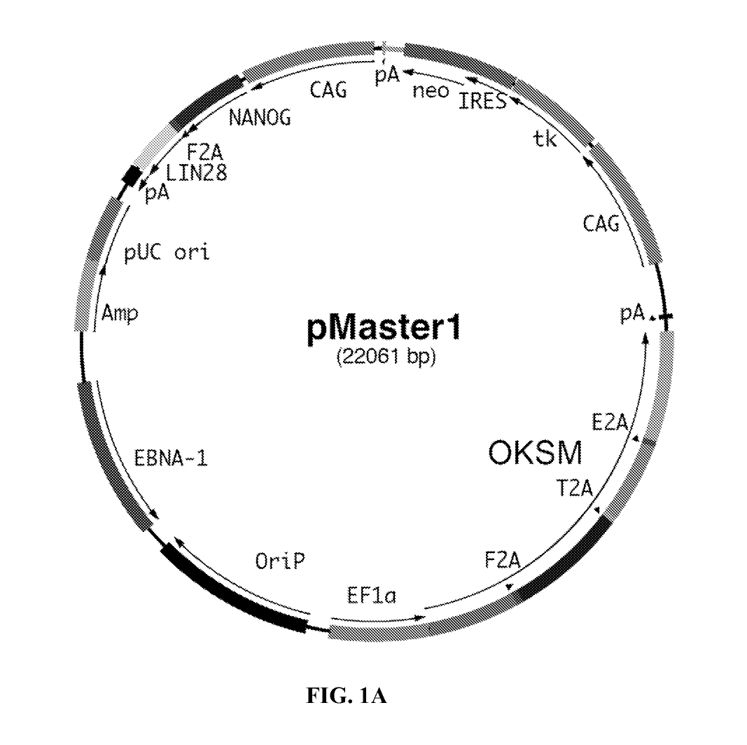

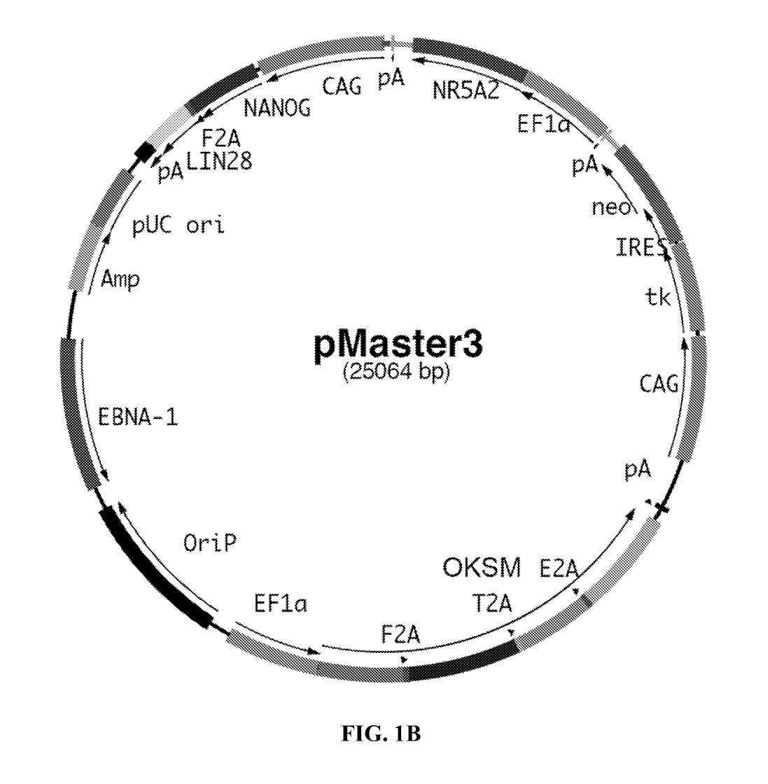

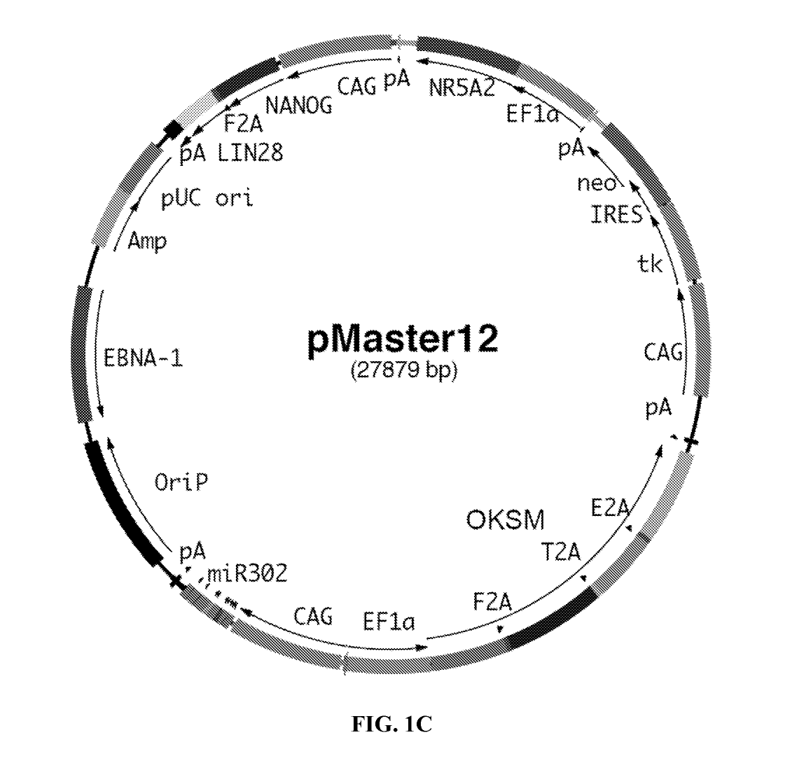

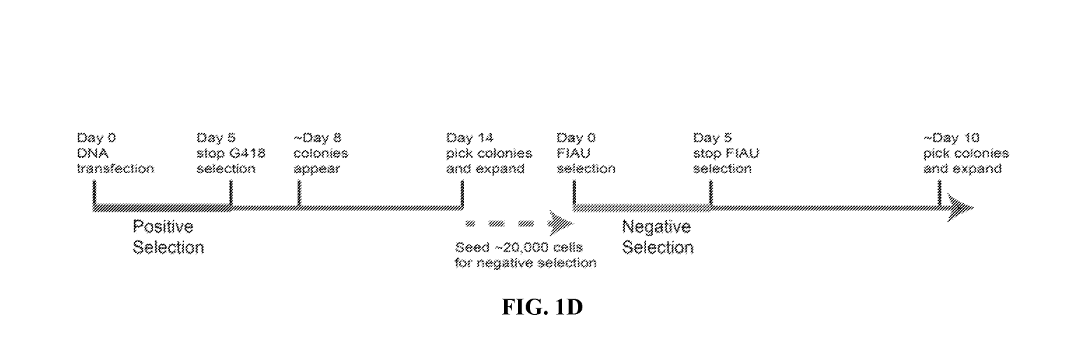

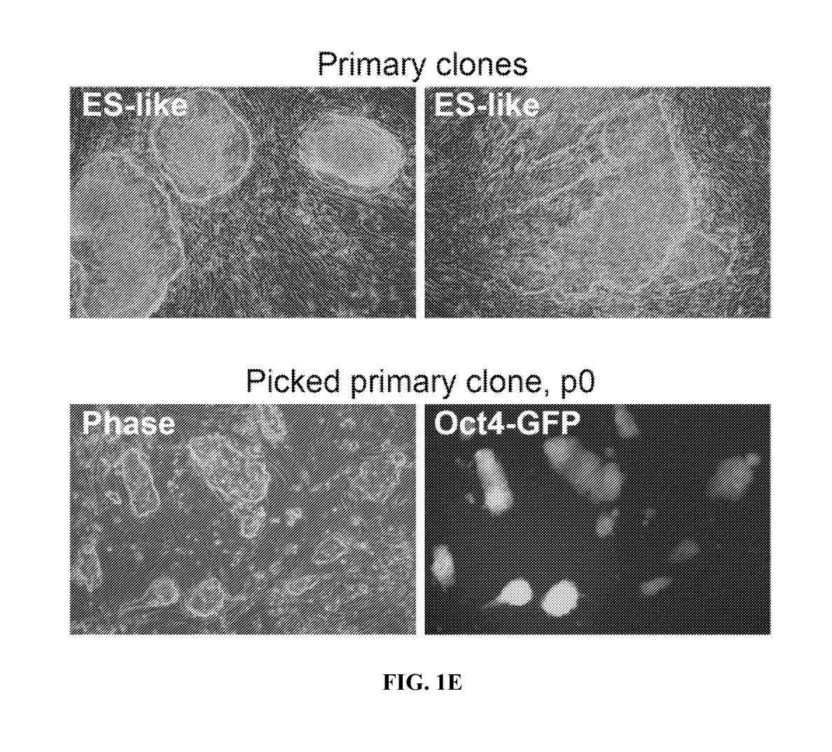

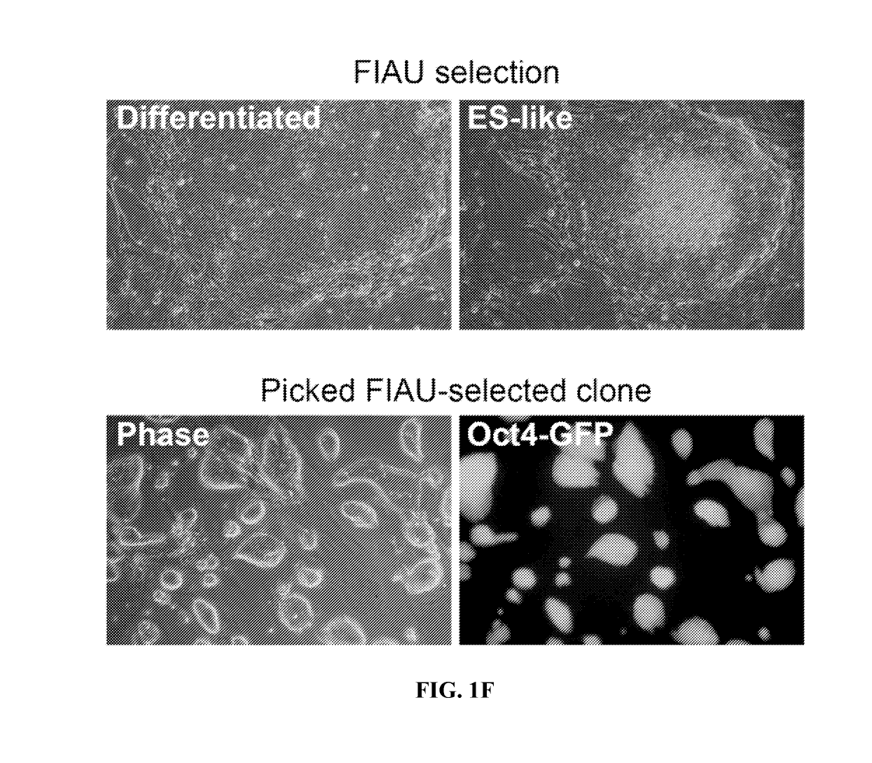

Three different episomes were generated and studied for their ability to yield transgene-free, germ line-competent iPS cells. Specifically, the four human genes OCT4, KLF4, SOX2 and cMYC linked by 2A sequences (the OKSM cassette), NANOG and LIN28, and neo and HSVtk were incorporated into pMaster1 (FIGS. 1A, 1B, and 1C). The pMaster3 episome vector was also constructed and contained, in addition to the genes present in pMaster1, the NR5A2 gene. Finally, to generate pMaster12, the microRNA 302/367 gene cluster was added to pMaster3.

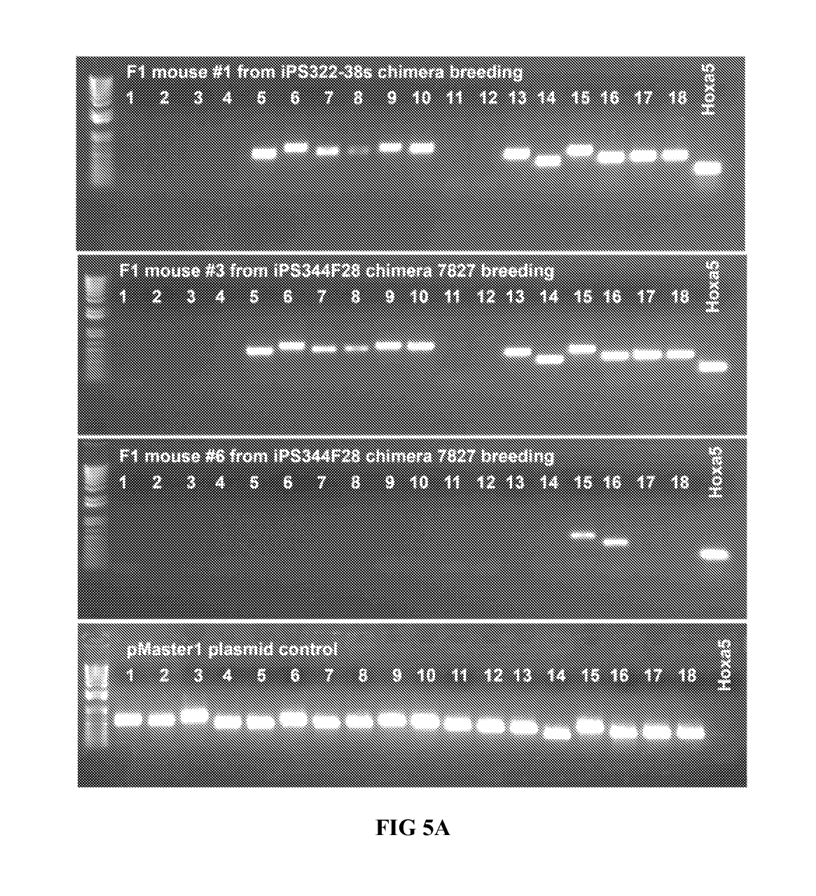

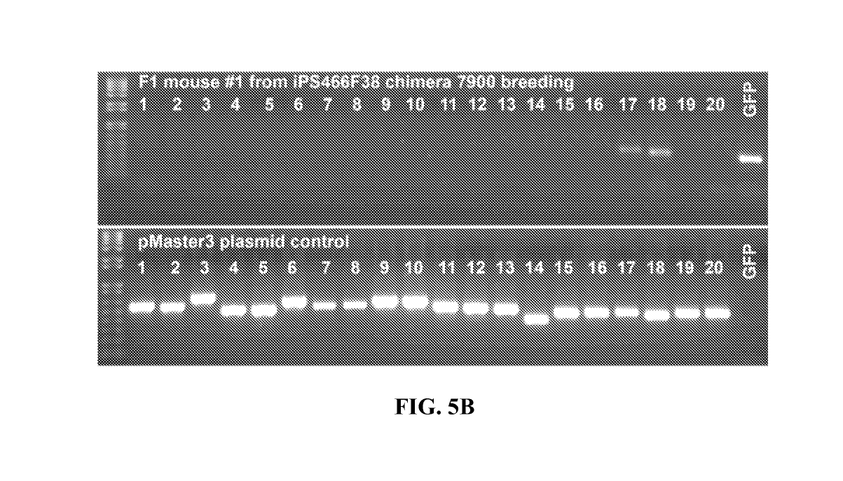

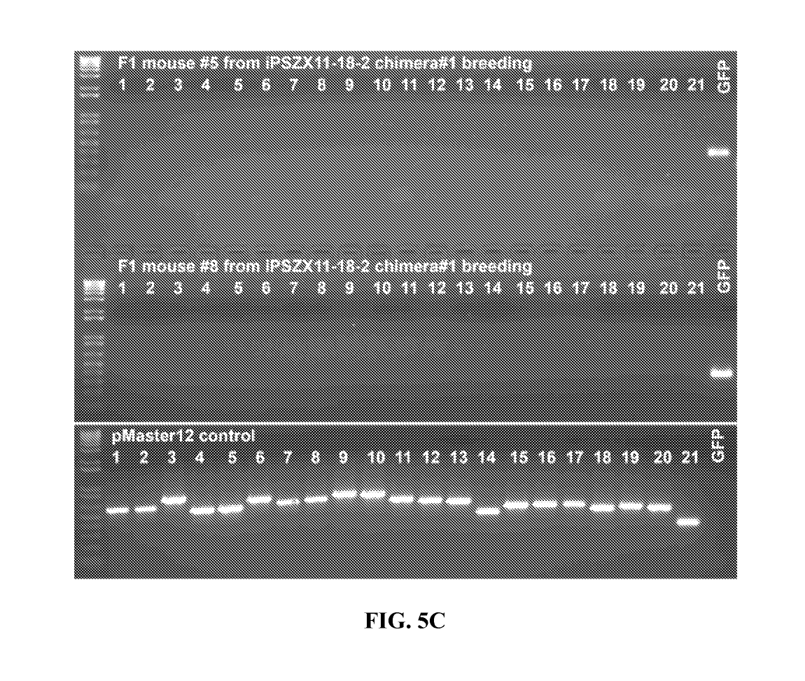

Introduction of the pMaster1 plasmid into Oct4-GFP MEFs generated iPS cells at an efficiency of about 0.01-0.02%. The iPS cell clones were readily expanded and showed GFP expression, indicating activation of the endogenous Oct4 gene (FIG. 1D). Primary iPS cells (passage 0) were seeded in FIAU containing media to select for cells that had lost the tk-bearing episome. On average, 0.01% of cells survived FIAU selection. About 25% of the clones picked following FIAU selection were sensitive to G418 implying they had also lost neo. PCR confirmed the absence of neo and HSVtk. Surprisingly, however, PCR analysis with 18 primer pairs covering the entire pMaster1 episome demonstrated persistence of the OKSM genes (FIG. 5A). The partial loss of transgenes from the episome indicated incomplete reprogramming using the pMaster1 episome for generation of iPS cells, thereby necessitating retention of the exogenous OKSM sequences.

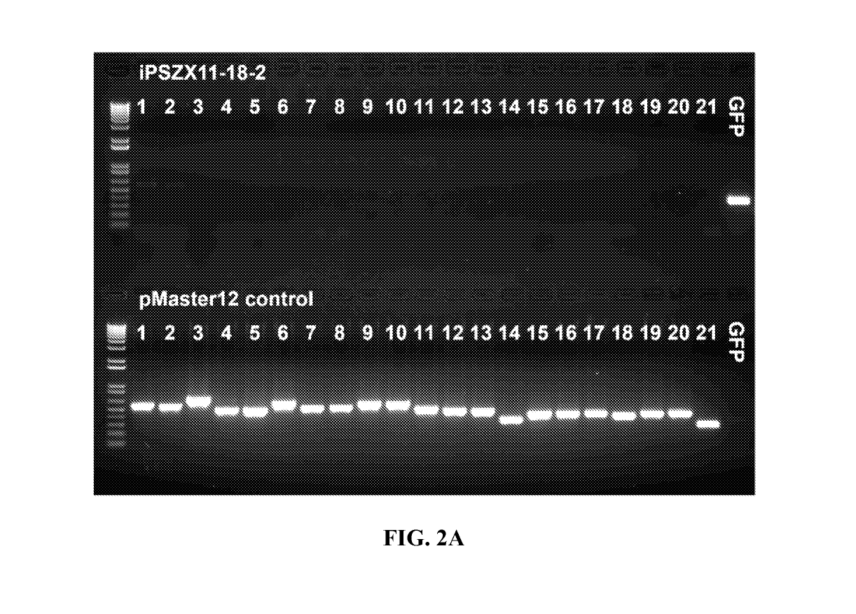

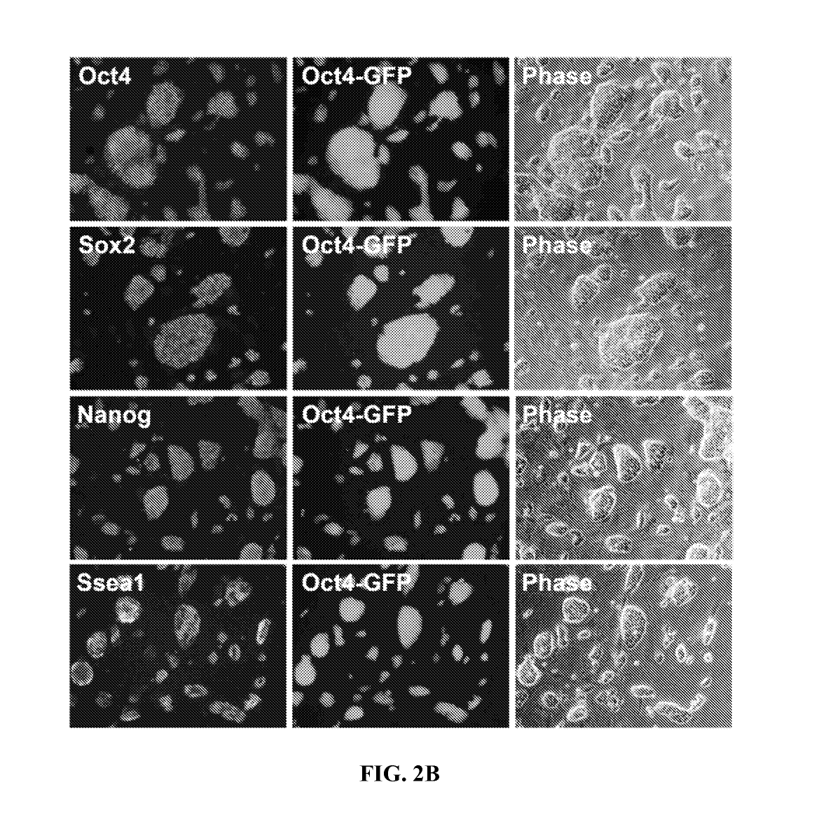

Next, pMaster3 and pMaster12 were evaluated. Slightly fewer colonies appeared on the primary culture plates following transfection of pMaster3, but these colonies displayed more compact mouse ES cell-like morphology (FIGS. 1E and 1F). With pMaster12, 10-fold more colonies appeared on the primary culture plates (efficiency, 0.20±0.03%). These primary pMaster12-derived iPS cells (9 lines tested) at passage 1 contained 4.3±3.0 copies per cell of the pMaster12 episome as determined by real-time PCR. When pMaster3- or pMaster12-derived clones were subjected to FIAU selection, approximately 25% of the primary clones produced G418-sensitive secondary clones. PCR analysis of pMaster3-derived clones showed persistence of the NR5A2 gene in some, but not all, of these clones (e.g. iPS466F38, FIG. 5B). PCR analysis of pMaster12-derived secondary clones with 21 primer pairs that covered the episome validated the complete removal of all of the episomal sequences in the majority (23 out of 30) of the G418-sensitive clones (FIGS. 2A and 5C).

Since the primary pMaster12 derived iPS cells at passage 1 contained only a few copies of the episome, the efficiency of deriving transgene-free clones by simple passaging, without using negative selection was examined. Among 12 primary clones tested, 2 clones became completely transgene-free at passage 7 as a whole plate without subcloning.

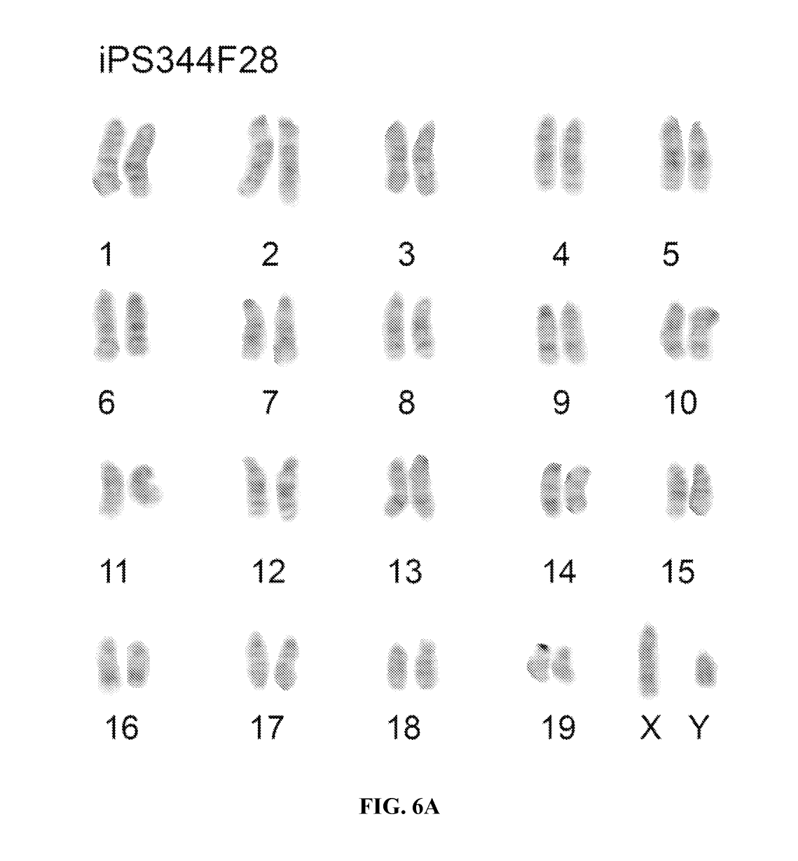

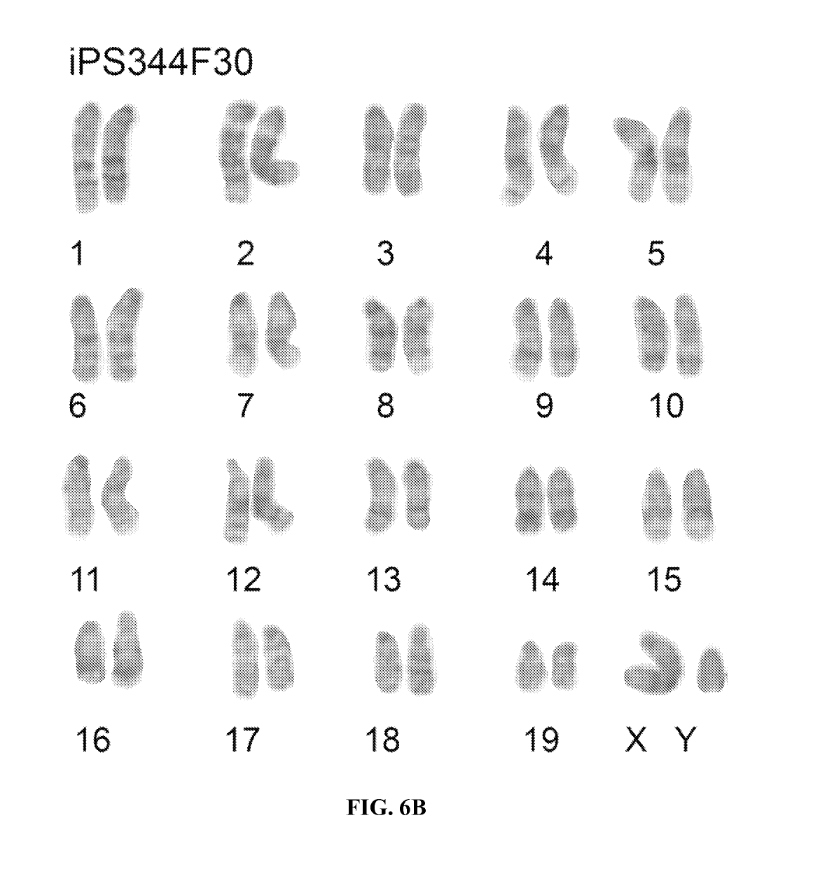

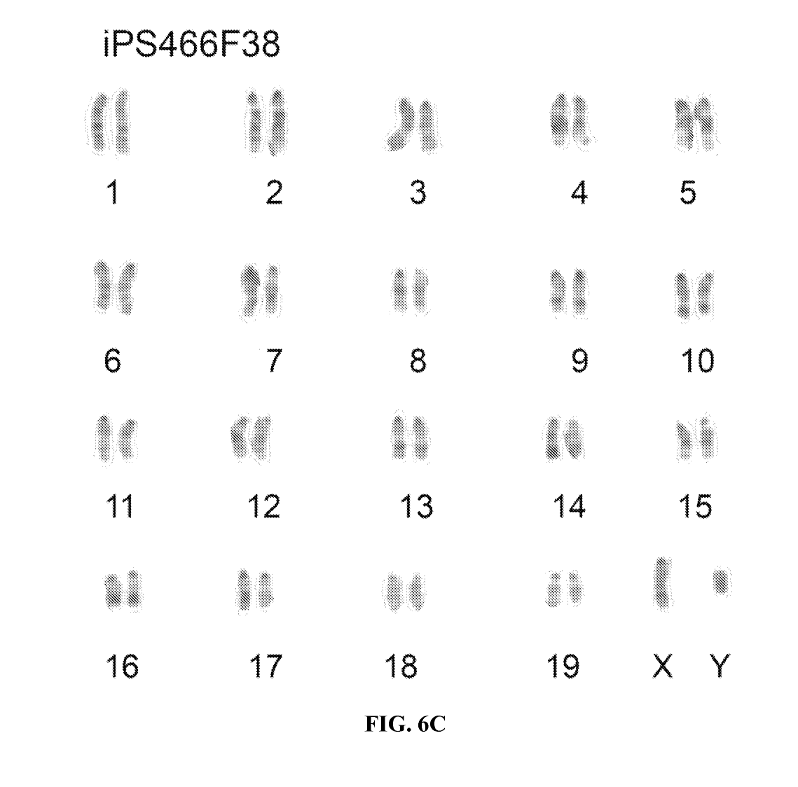

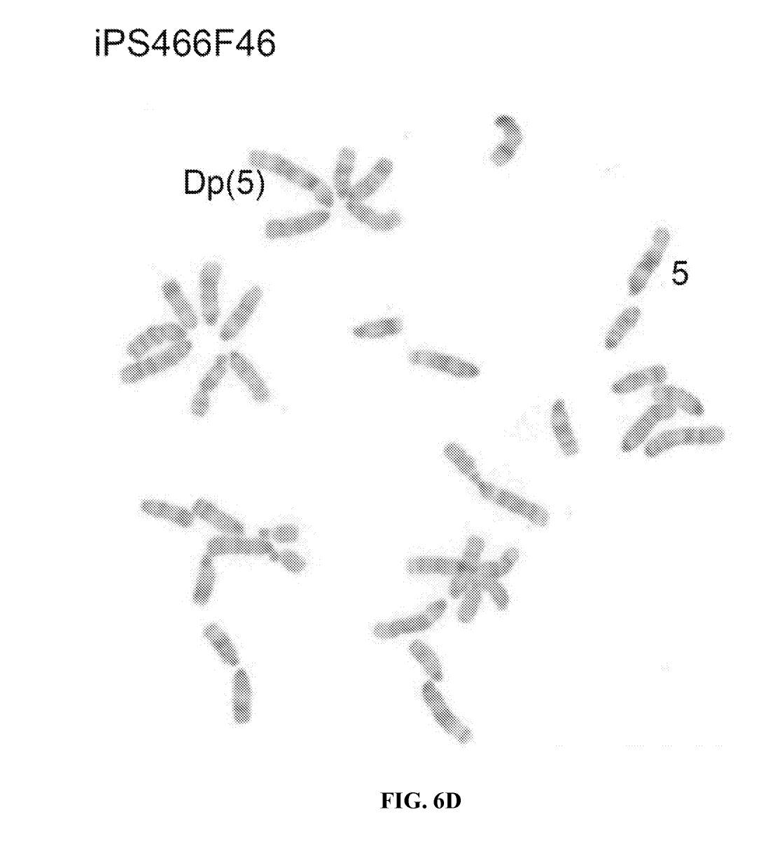

Immunostaining of pMaster3- and pMaster12-derived transgene-free iPS cell lines, such as iPSZX11-18-2, showed homogeneous expression of endogenous pluripotency markers FIG. 2B). iPS cell lines derived using pMaster episomes demonstrated normal karyotypes with the exception of iPS466F46 (FIGS. 2C and 6). Some were maintained for more than 35 passages without detectable changes in karyotype, morphology or growth characteristics.

To test the ability of pMaster-derived iPS cell lines to generate germ line chimeras, blastocyst and eight-cell morula injections were performed. Five lines grown in conventional ES cell media generated several low-percent coat color chimeras and one high-percent coat color chimera (Table 1). None of these chimeras showed germ line transmission after extensive breeding.

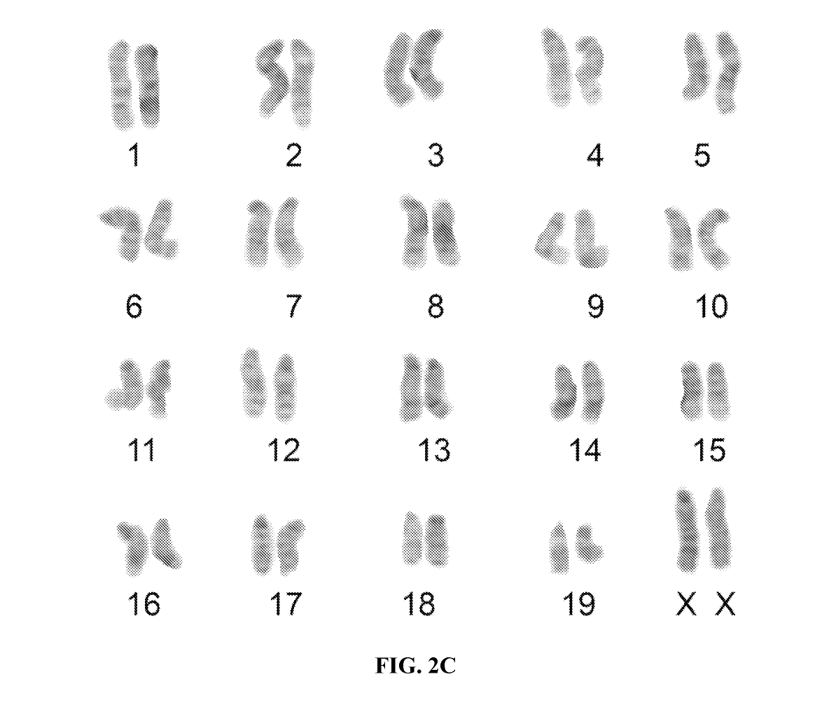

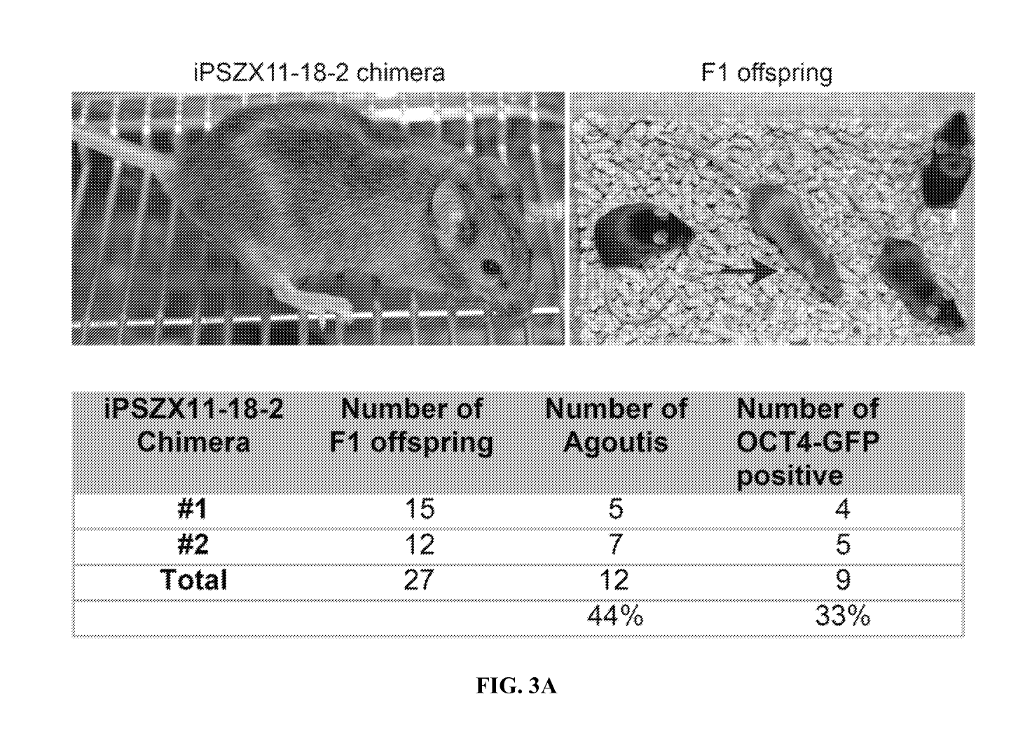

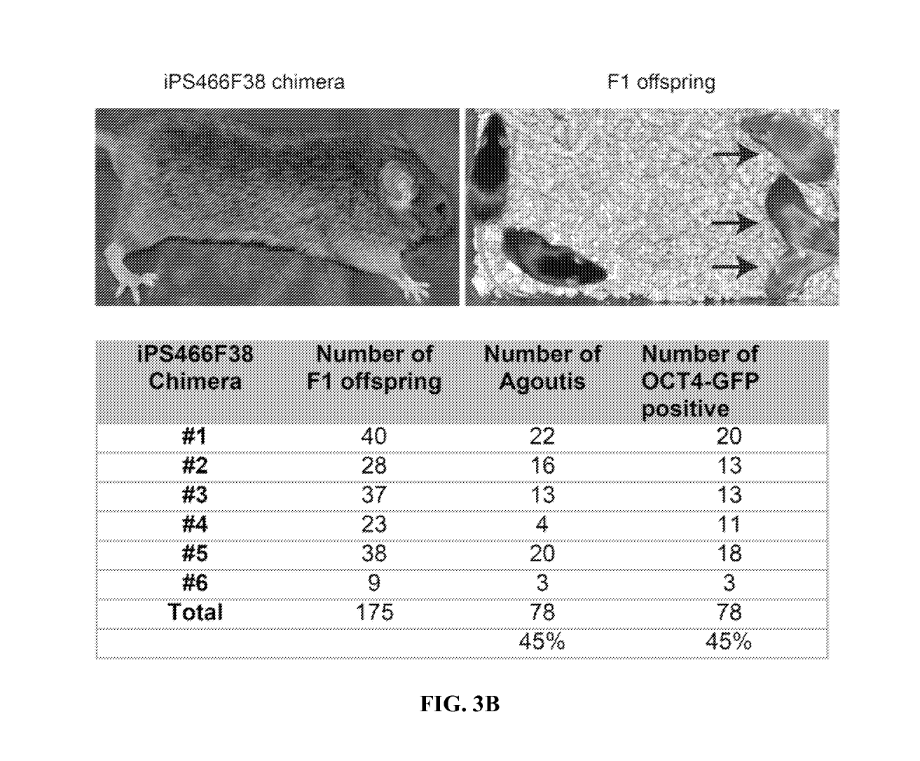

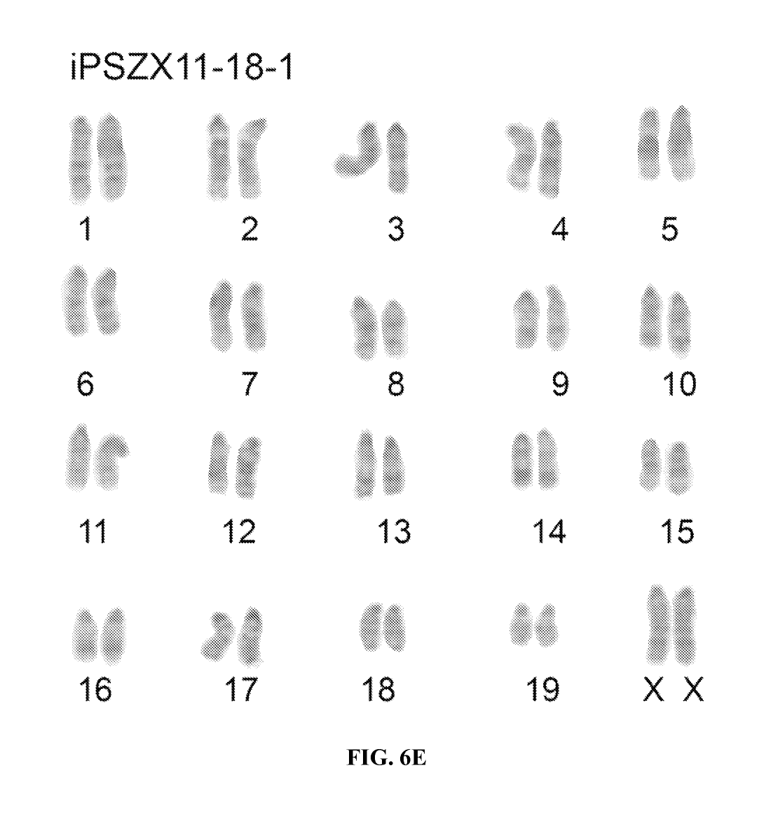

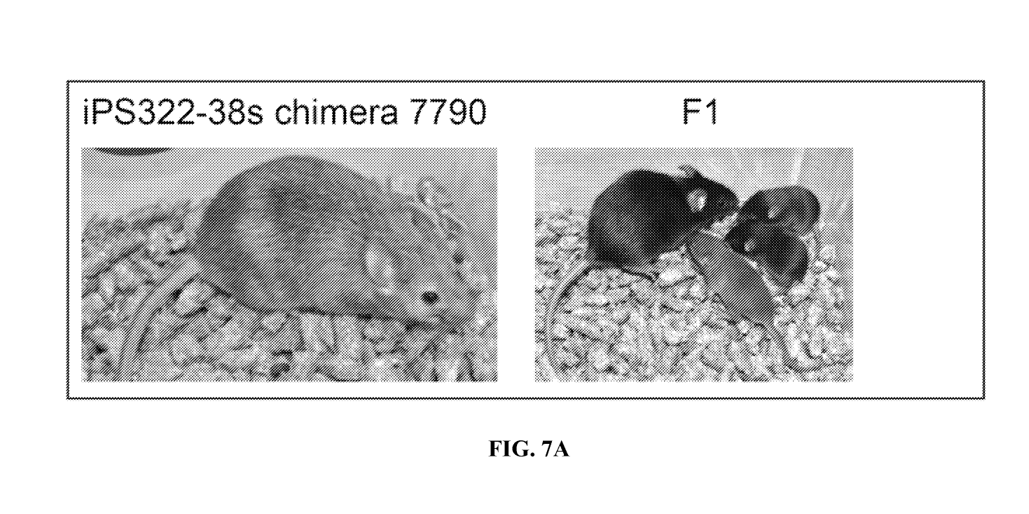

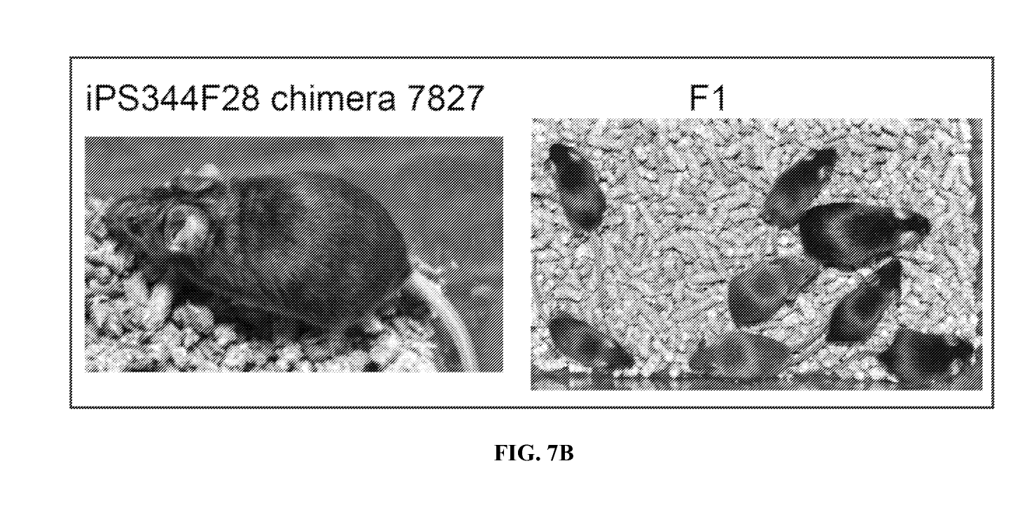

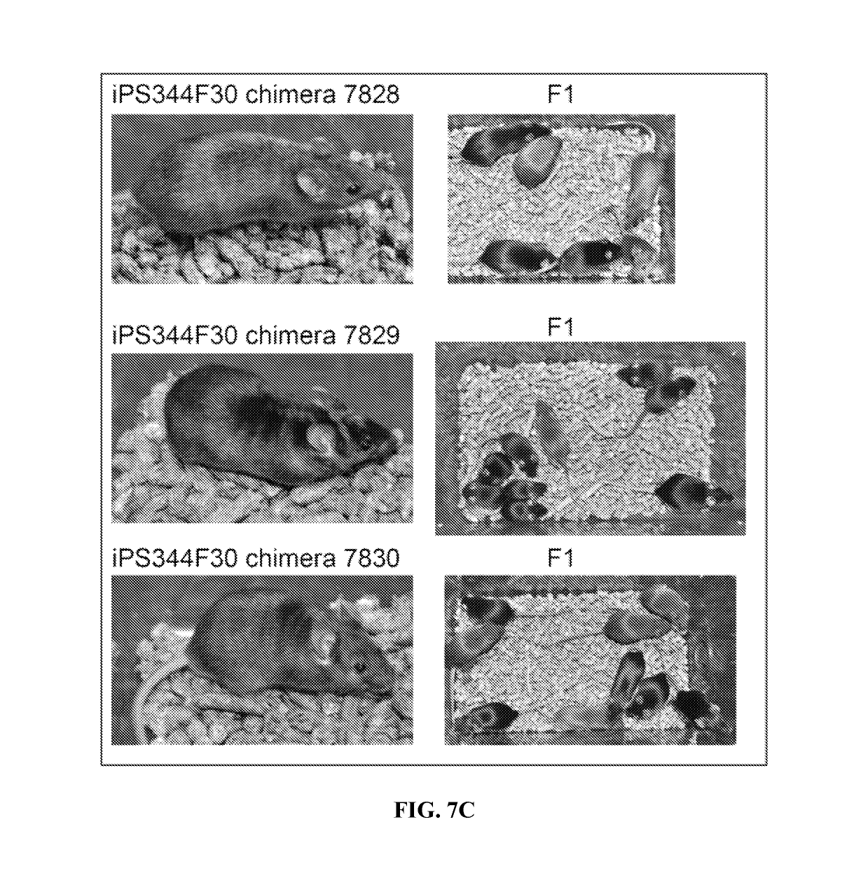

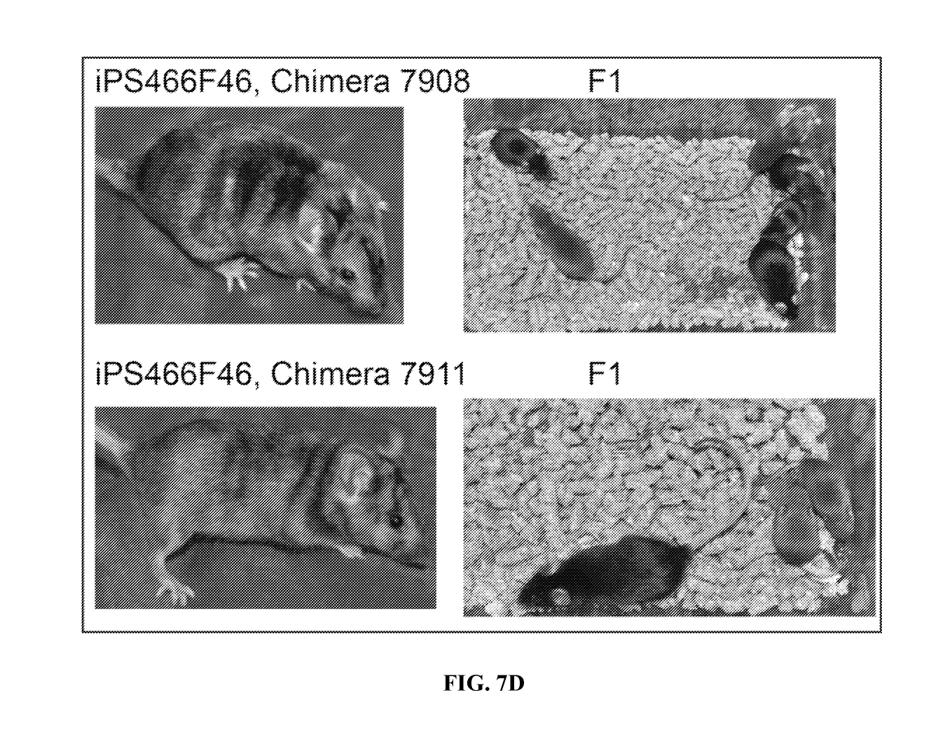

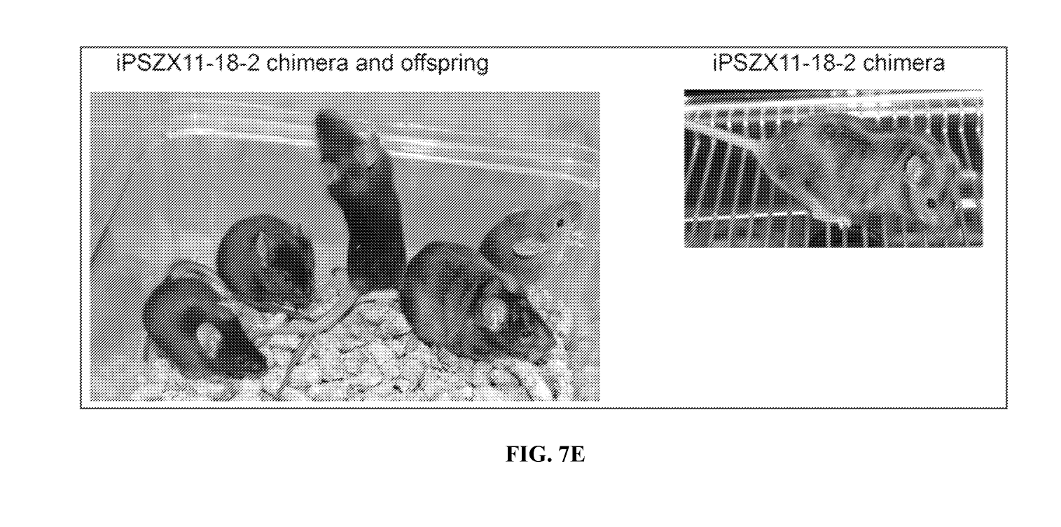

2i media, containing small molecule inhibitors of the MEK/ERK and GSK3b pathways, promoted cells to a more pluripotent state. The germ line competency of pMaster1-derived cell lines (iPS322-38s, iPS322-40t, iPS344F28 and iPS344F30), pMaster3-derived cell lines (iPS466F38 and iPS466F46), and pMaster12-derived cell lines (iPSZX11-18-1 and iPSSZX11-18-2) grown in 2i media were tested. All eight lines produced high percentage chimeras and 7 generated germ line chimeras (Table 2 and FIG. 7). For example, iPSZX11-18-2 cells produced 5 females that were more than 50% chimeric by coat color (FIG. 3, Table 2). Matings to wild type males transmitted the iPS-derived Oct4-GFP allele to more than 33% of progeny (since Oct4-GFP was a heterozygous allele, the maximum possible transmission frequency was 50%). Extensive PCR analysis of offspring confirmed the production of offspring lacking episome-derived reprogramming genes (FIG. 5C). These results illustrated that transgene-free iPS cells exhibited germ line competencies comparable to commonly used mouse ES cell lines (e.g., R1 and G4).

2i medium significantly improved the attainment of germ line competency for pMaster-derived iPS cells. To explore the mechanism of how 2i medium improved iPS cell quality, genome-wide microarray analysis of seven iPS cell lines (pMaster1: iPS322-38s, iPS322-40t, iPS344F28 and iPS344F30; pMaster3: iPS466F38 and iPS466F46), and two ES cell lines (R1 and G4), cultured in defined 2i medium or serum containing medium was performed. All of the pMaster1-derived cell lines retained the 6 transgenes (OKSM, NANOG and LIN28). The pMaster3 line iPS466F46 was transgene-free, and iPS466F38 retained NR5A2 but not the other transgenes. All of the lines analyzed except iPS466F46 exhibited normal karyotypes. Six of those pMaster1- or pMaster3-derived cells (Table 2) grown in 2i and used for blastocyst injection gave rise to germ line chimeras. All of the iPS and ES cell lines analyzed were male lines.

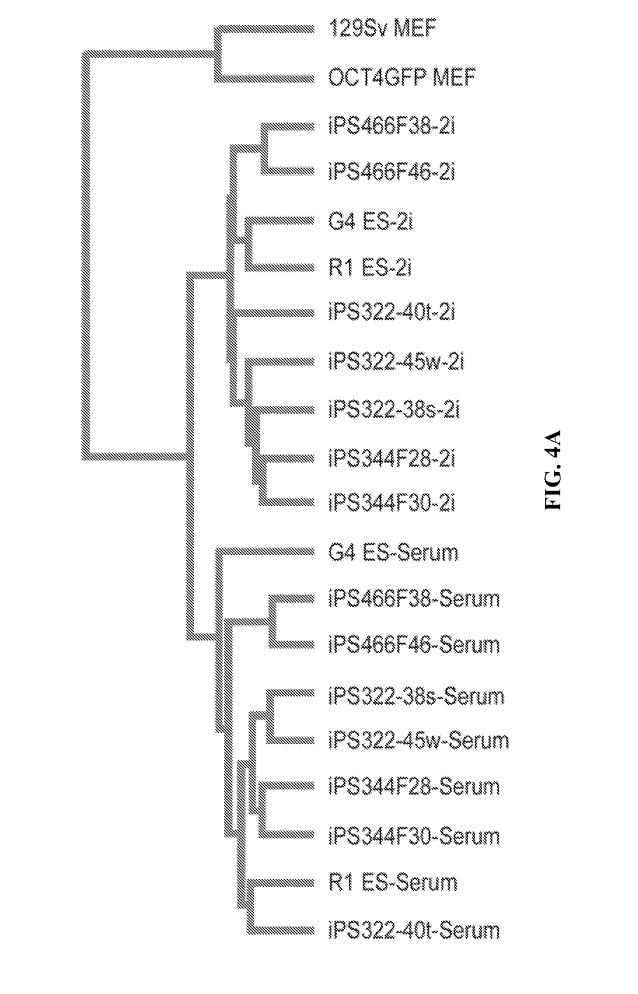

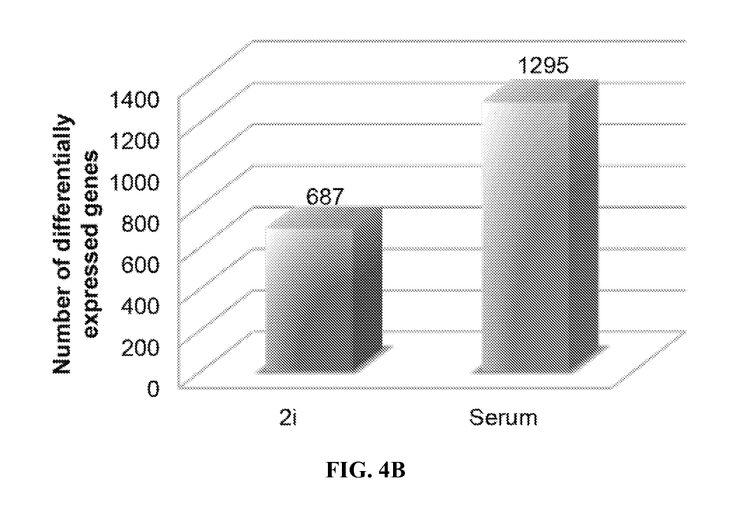

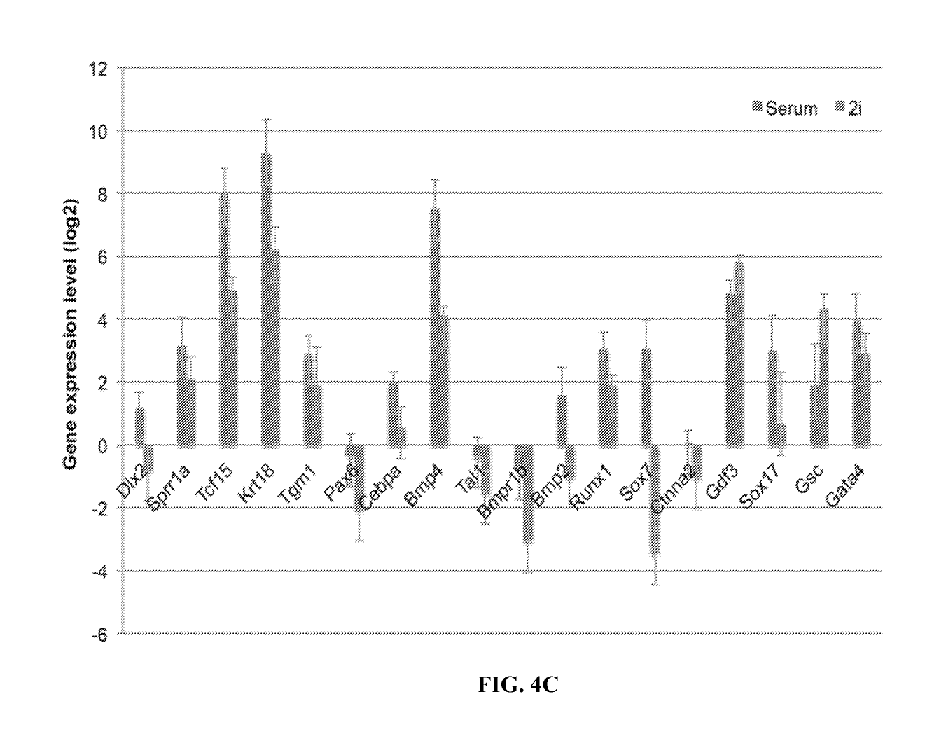

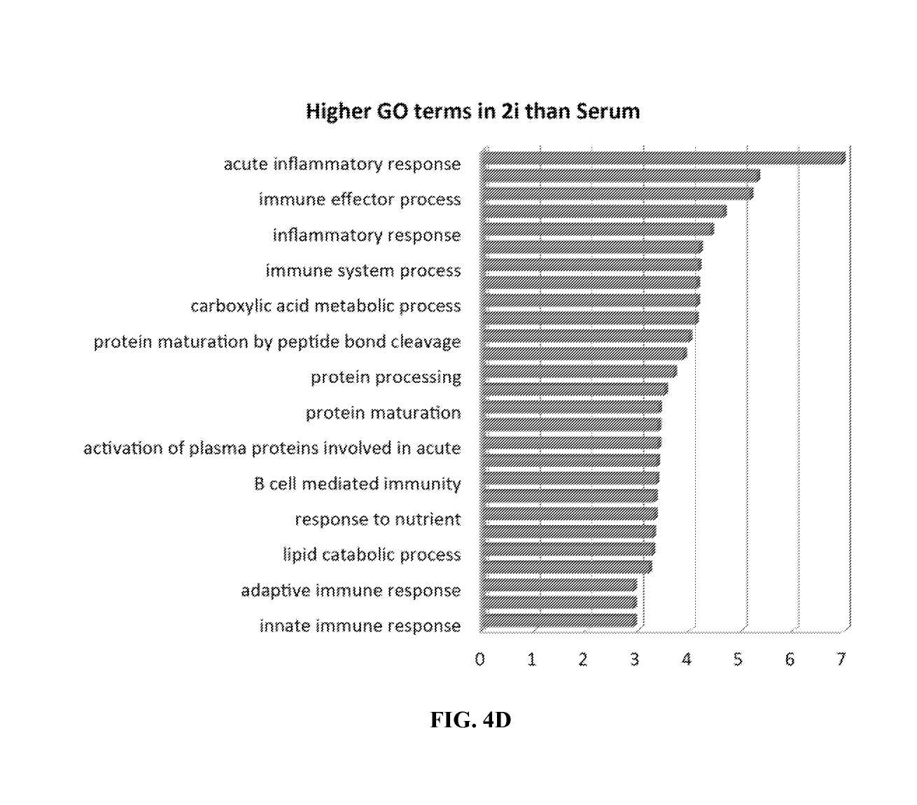

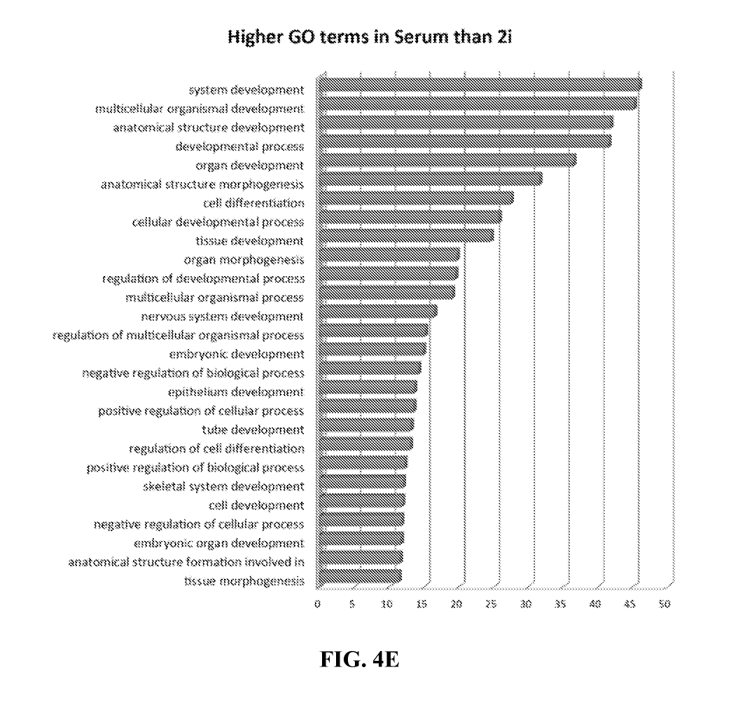

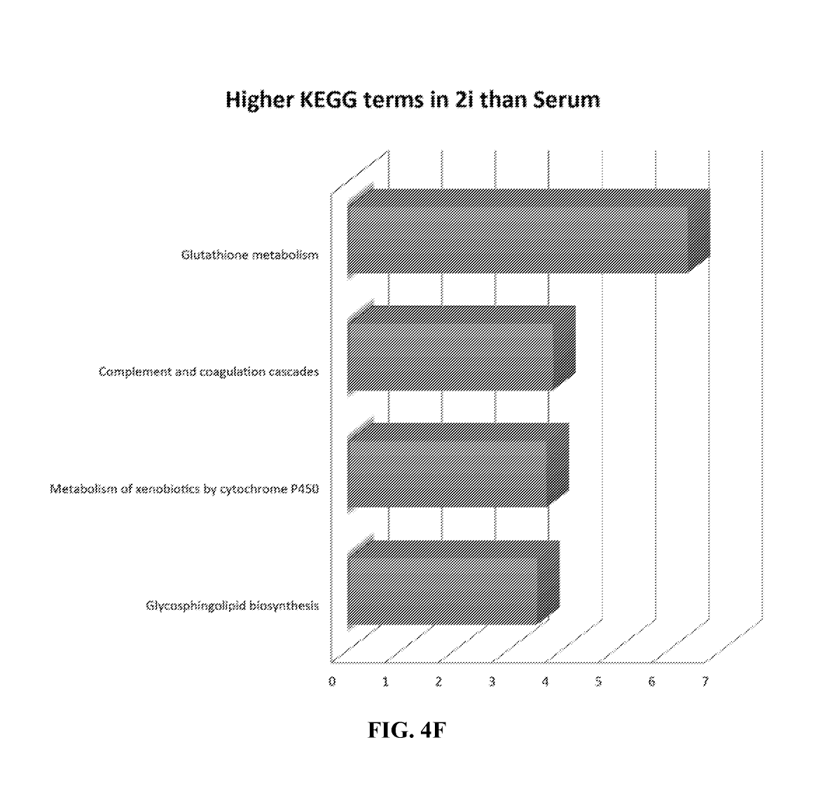

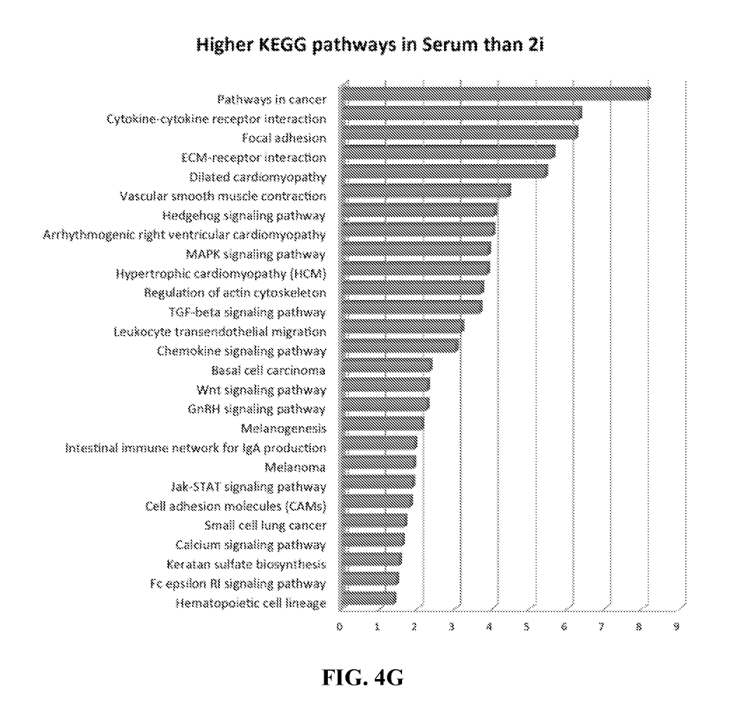

Microarray expression analysis revealed that iPS cells grown in 2i resembled more closely ES cells grown in 2i, whereas iPS cells cultured in serum resembled more closely ES cells cultured in serum (FIG. 4A). Pair-wise comparison analysis revealed that 687 genes expressed more than 2-fold higher in defined 2i medium, while 1295 genes expressed more than 2-fold higher in serum containing medium (FIG. 4B). Typical marker genes related to the differentiation of the three germ layers were activated in serum containing medium compared to 2i medium (FIG. 4C). To functionally annotate differentially expressed genes for iPS cells grown in 2i medium vs. serum media, these genes were analyzed through GO (Gene Ontology) terms and KEGG (Kyoto encyclopedia of genes and genomes). Genes higher in 2i (FIGS. 4D, 4E, 4F, and 4G) were highly enriched for terms of the innate immune response, which may lower the obstacles to reprogramming It was also apparent that 2i medium greatly inhibited expression of genes related to cell differentiation, tissue and organ development, indicating more effective maintenance of the iPS cells in an undifferentiated “ground state” (FIGS. 4C, 4D, 4E, and 4F). The results of inhibition of differentiation genes and enhanced expression of genes of the innate immune response system observed from growth in 2i medium relative to serum containing medium accounted for the marked improvement of iPS quality, with respect to germ line competence.

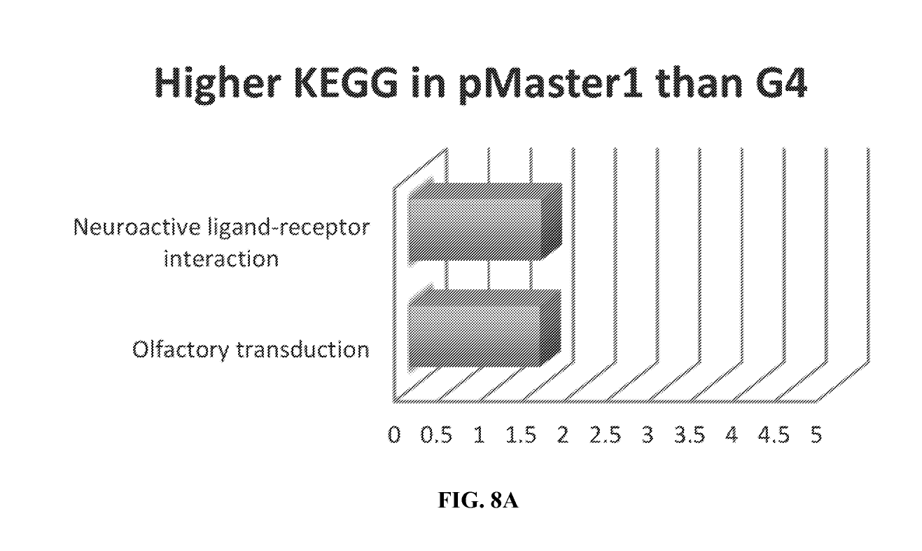

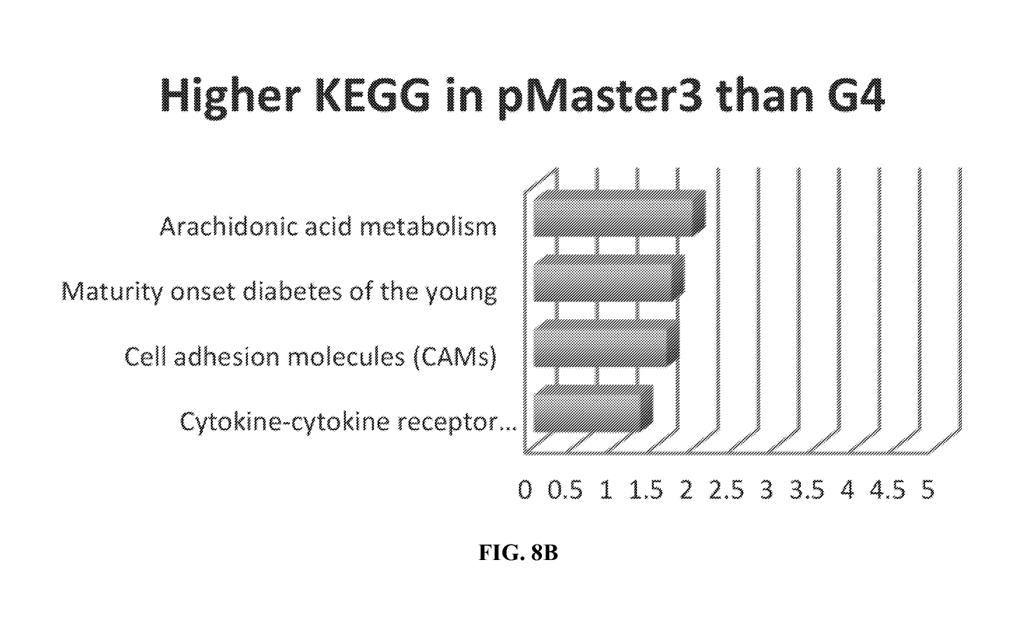

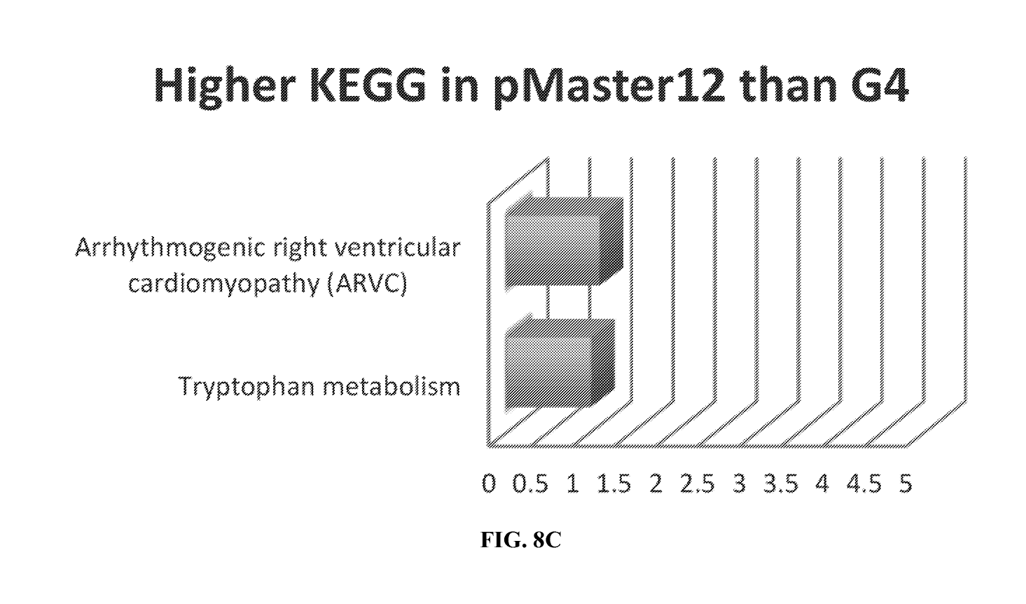

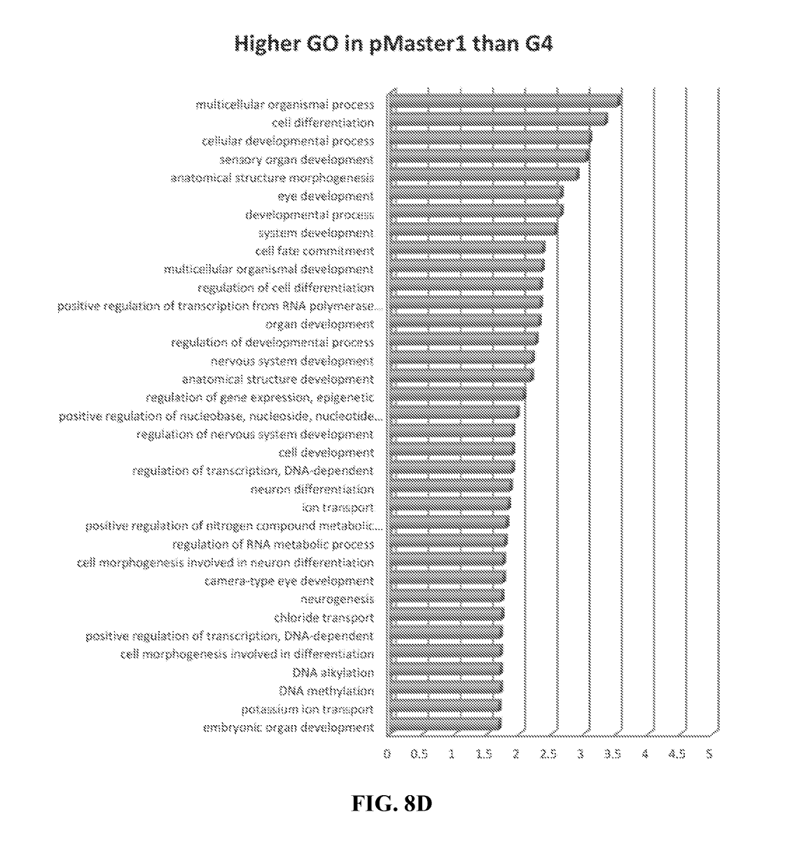

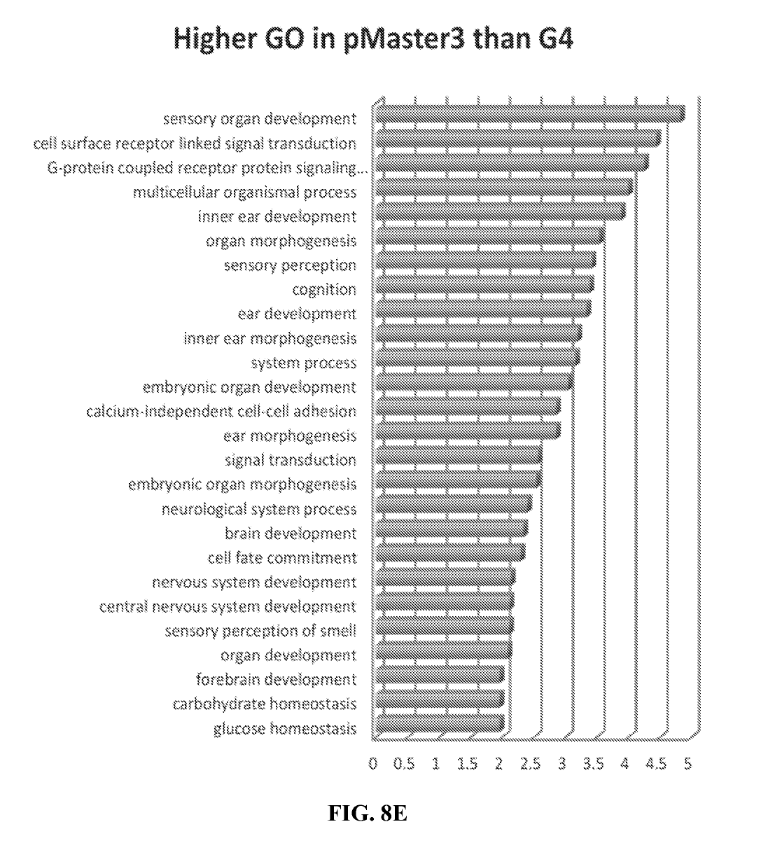

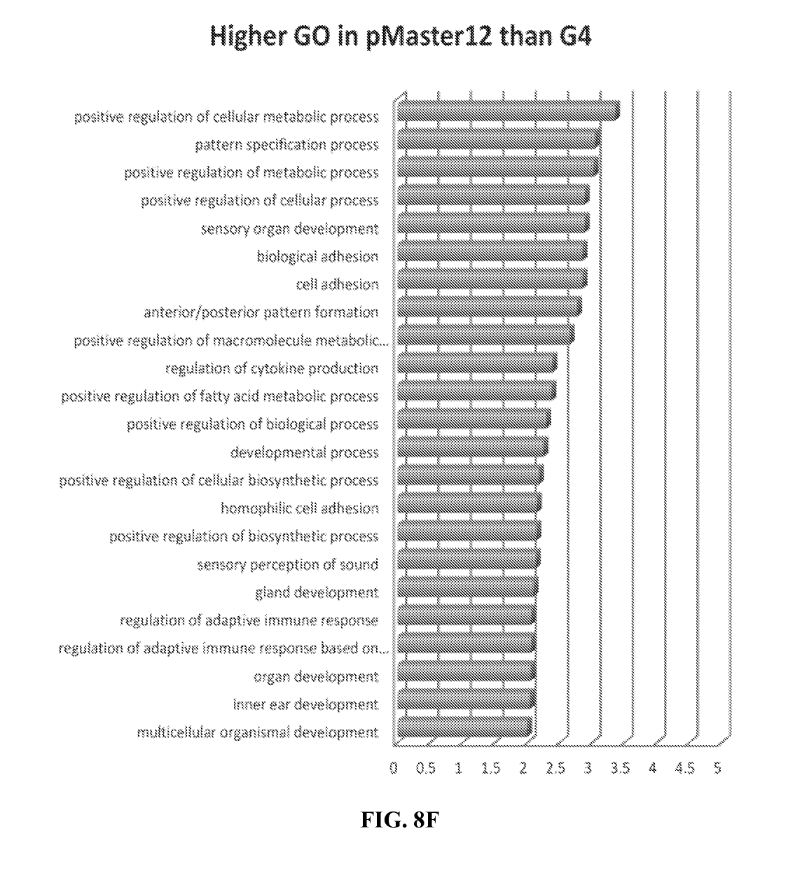

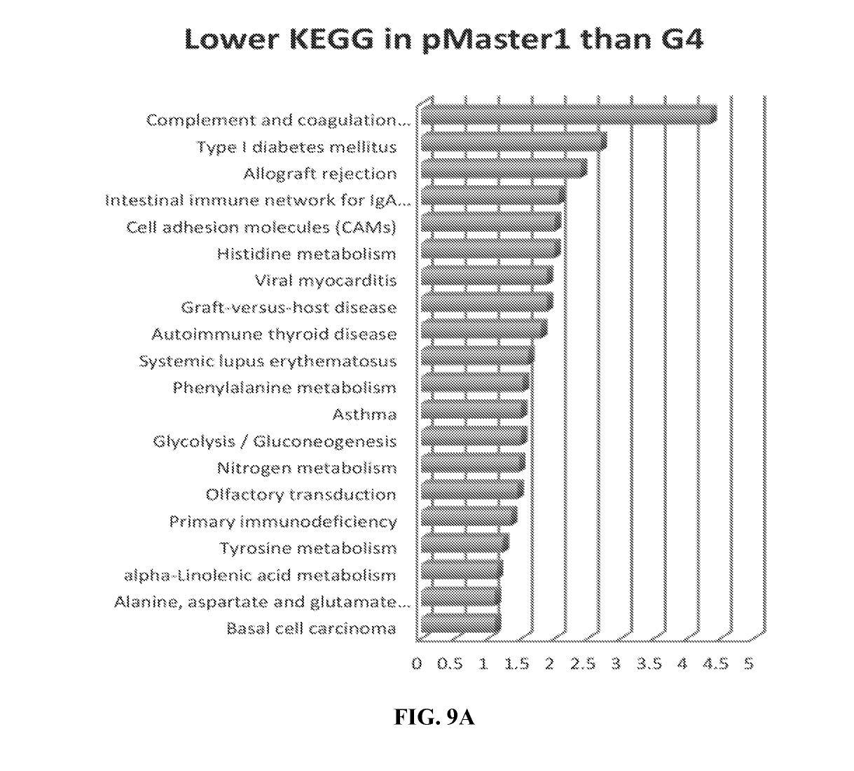

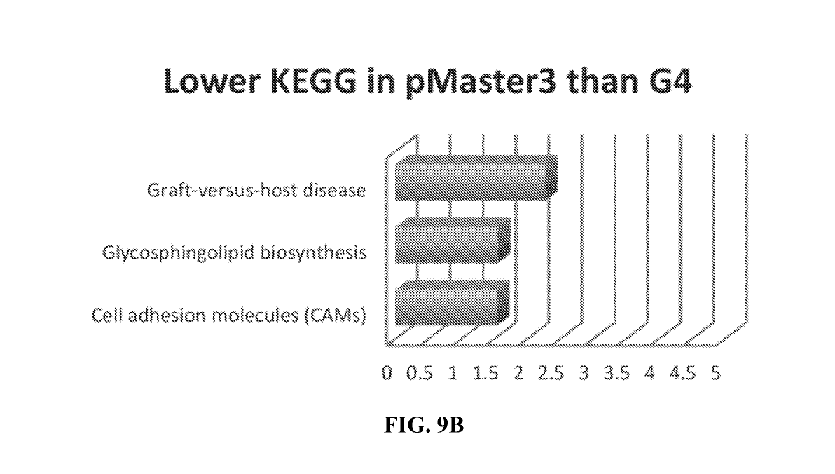

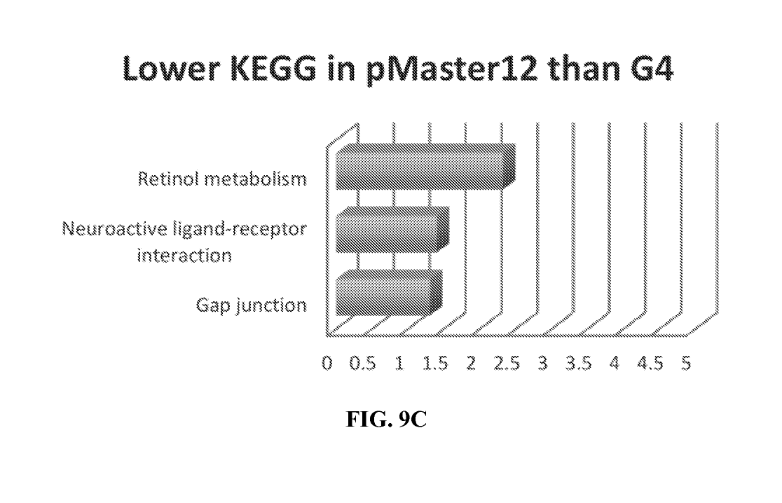

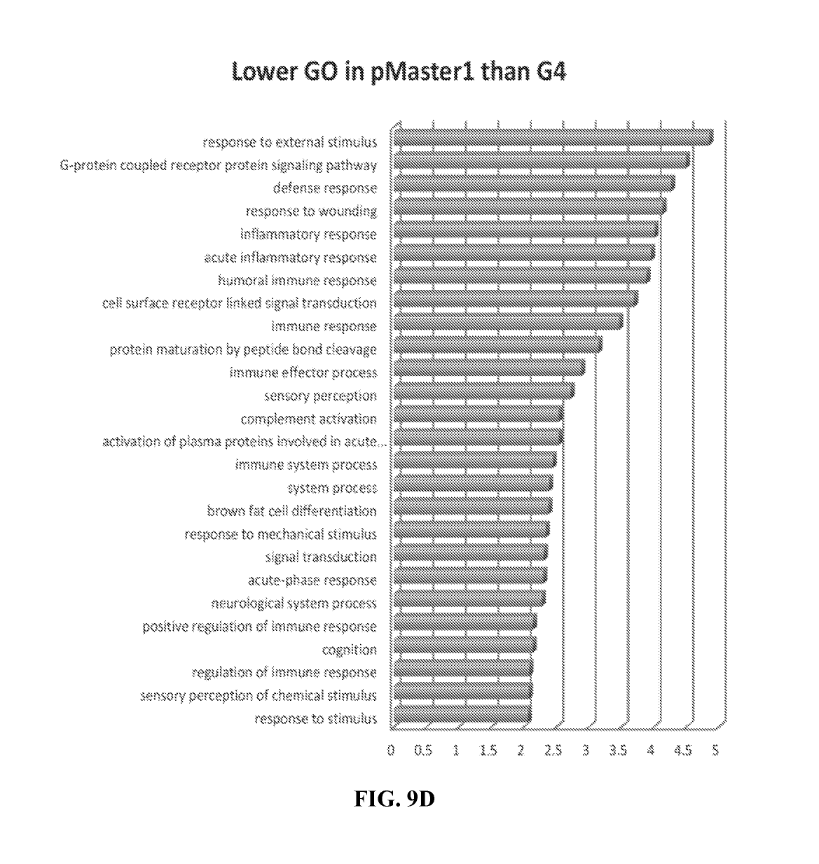

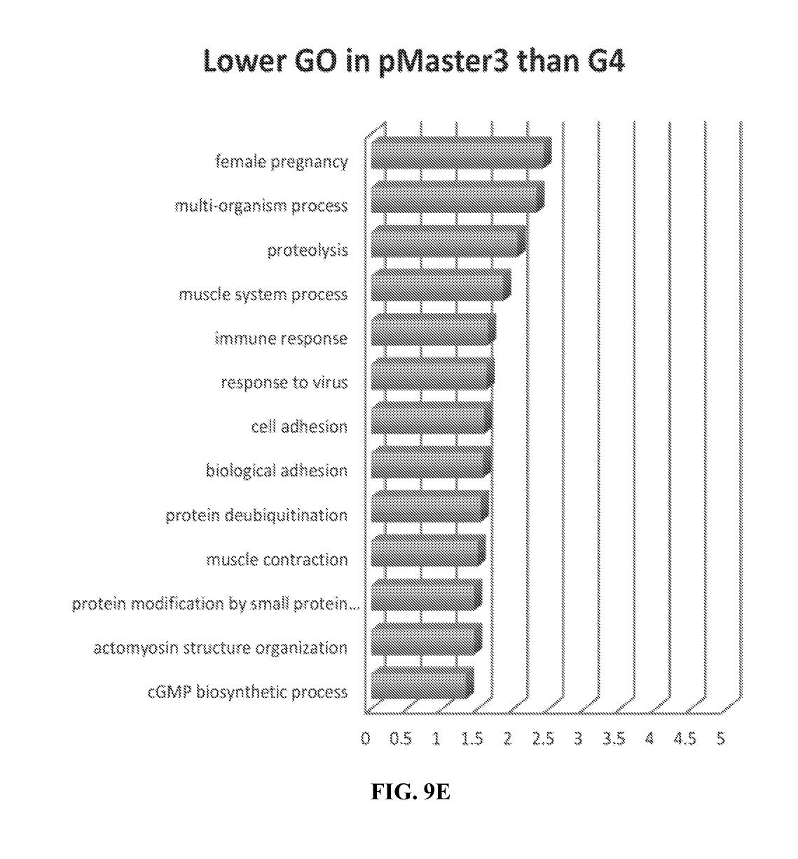

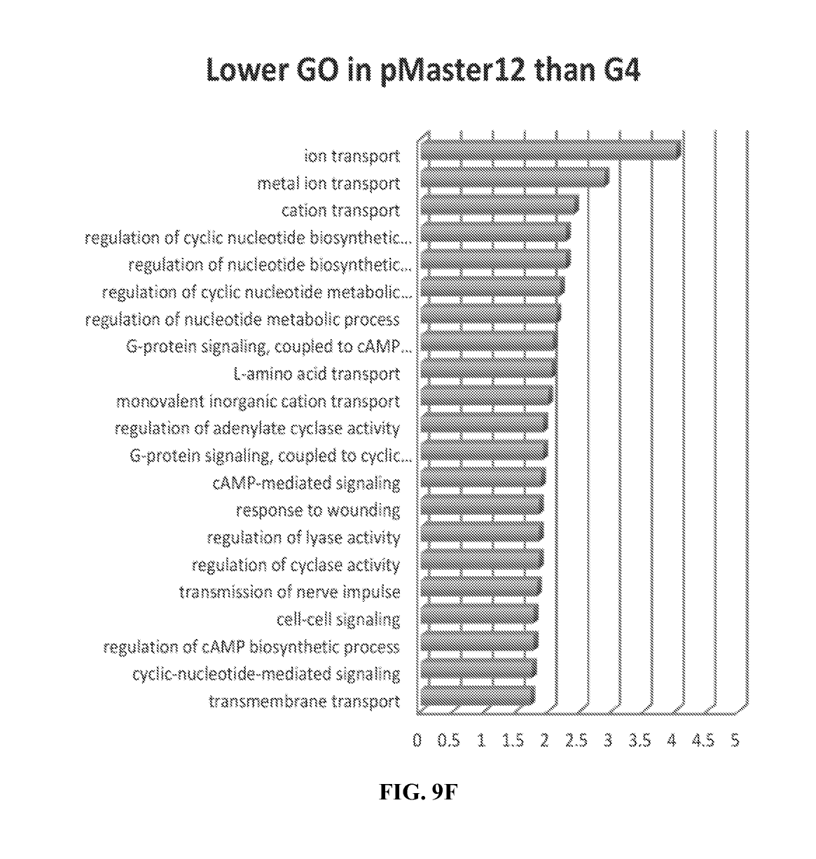

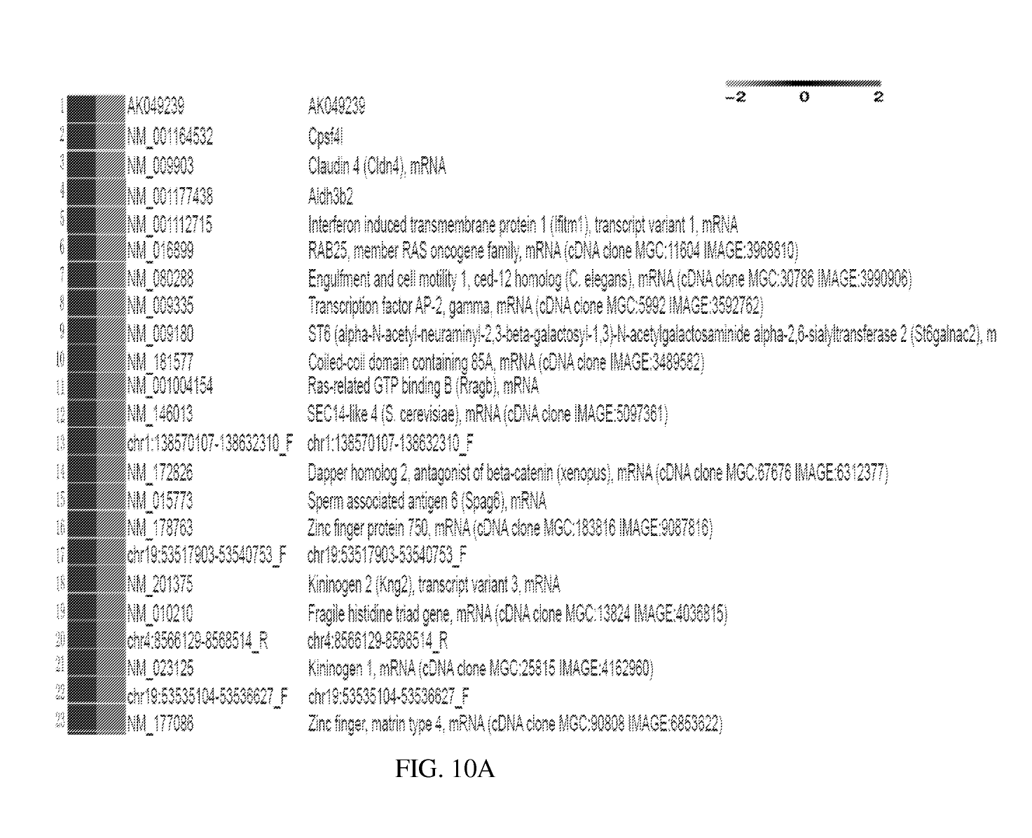

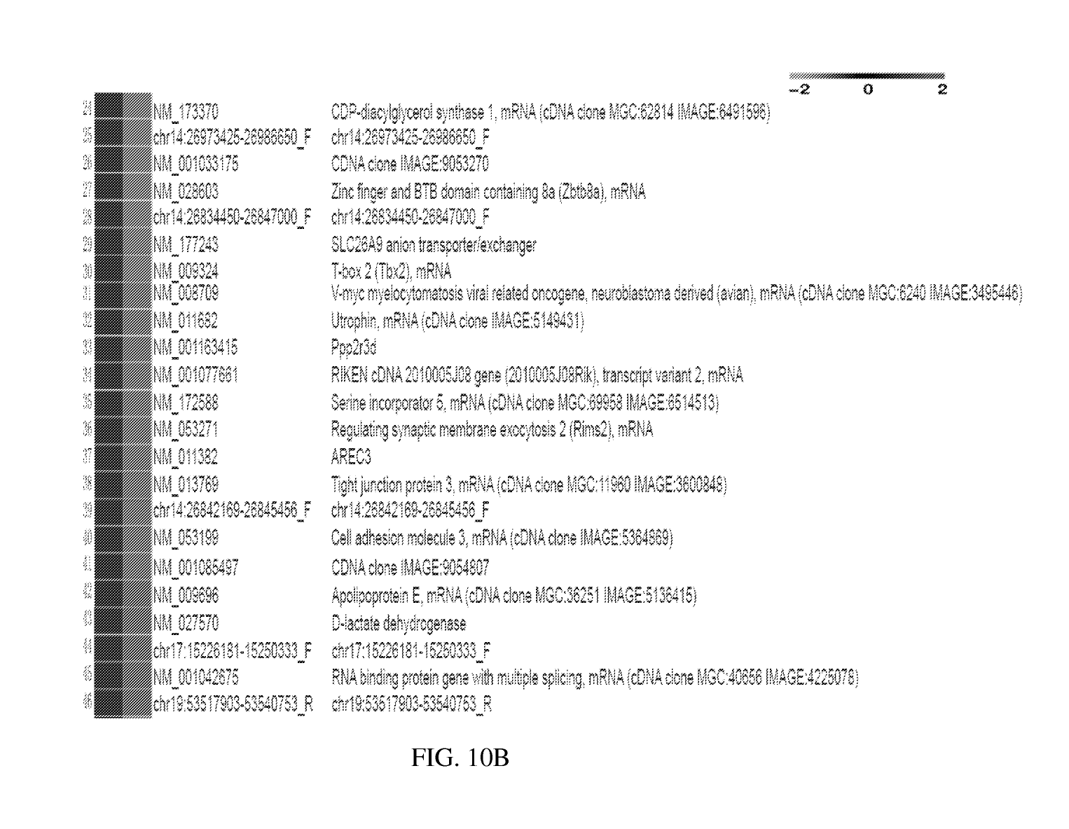

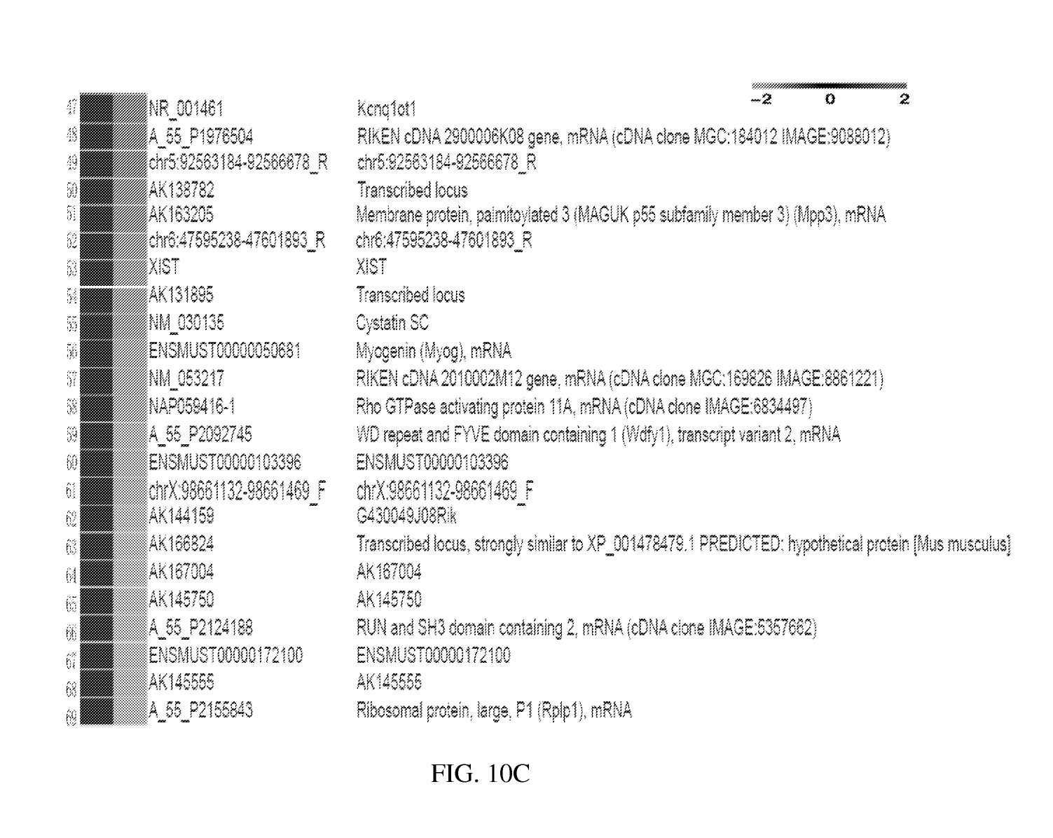

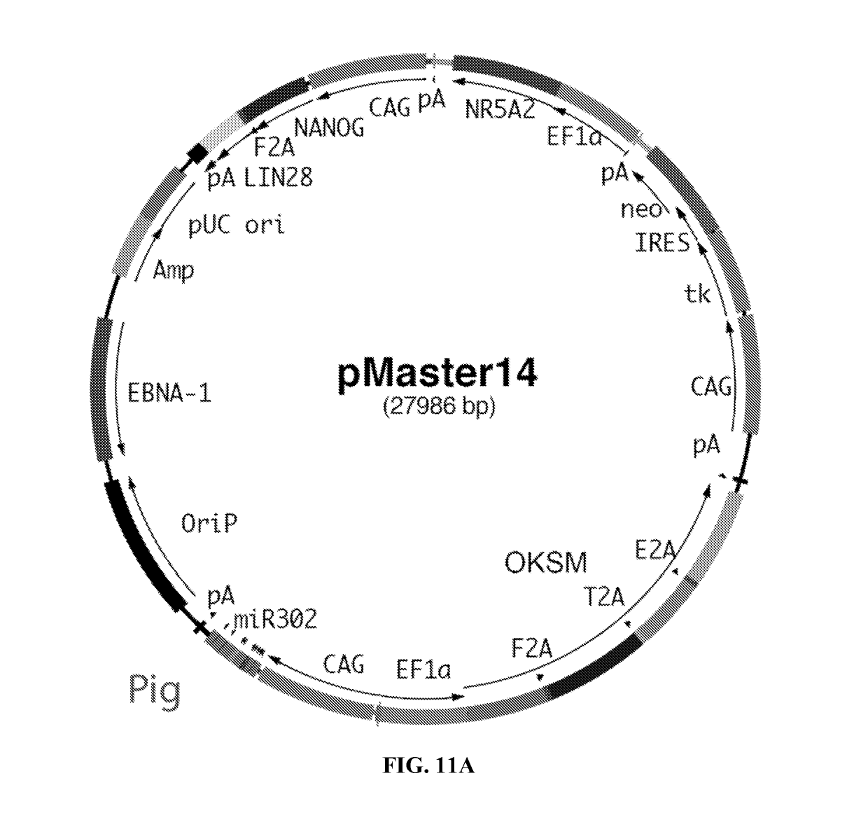

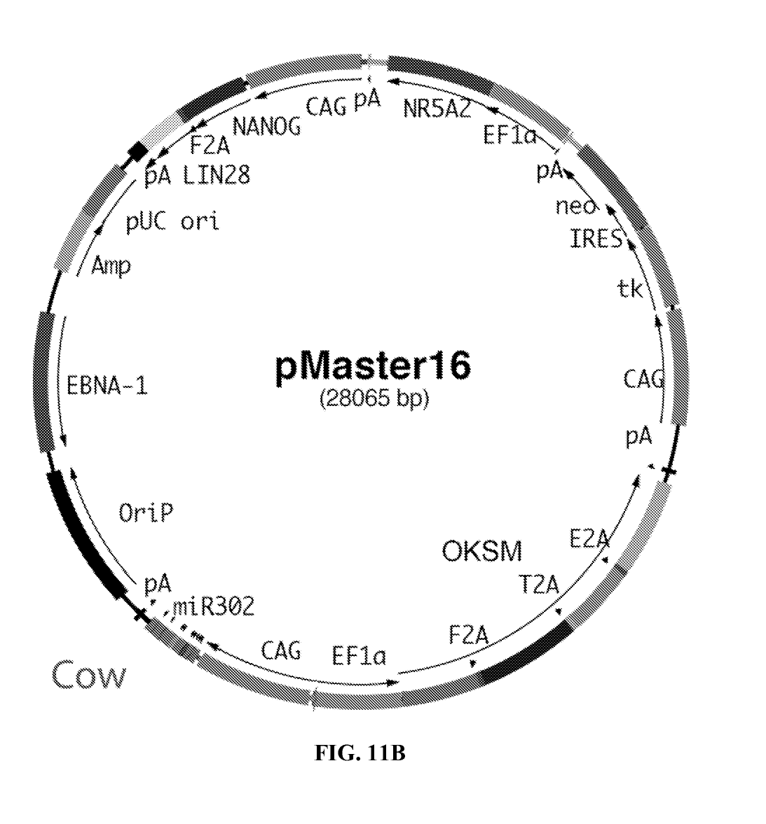

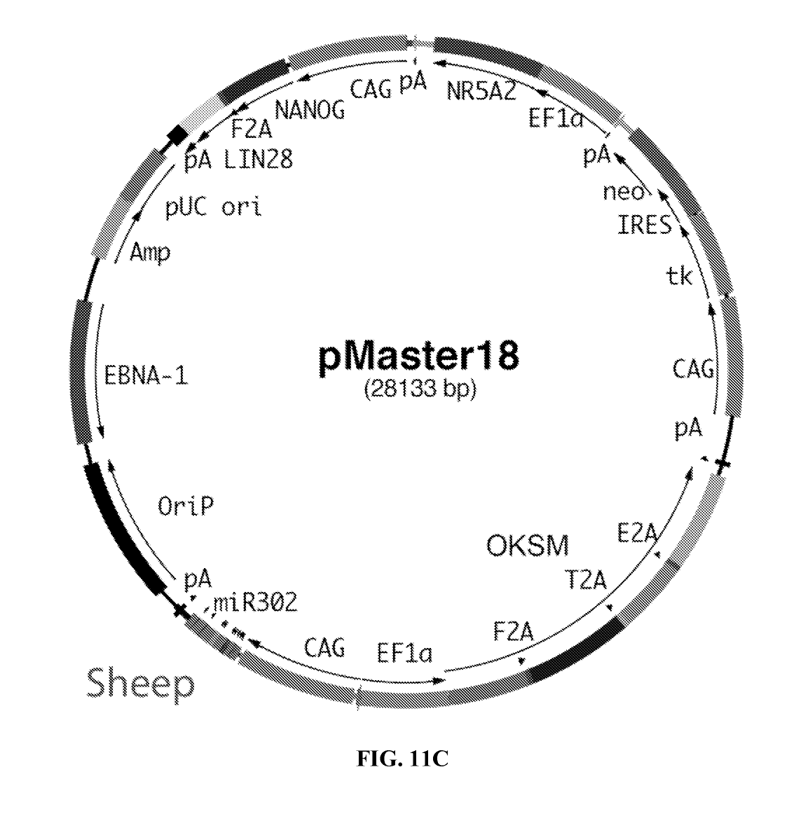

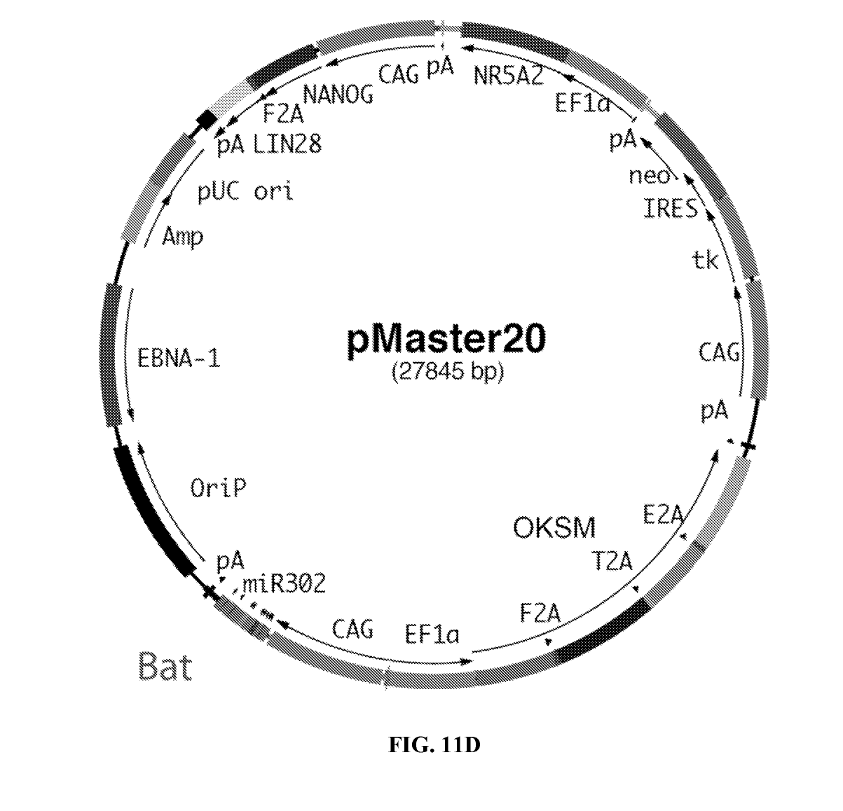

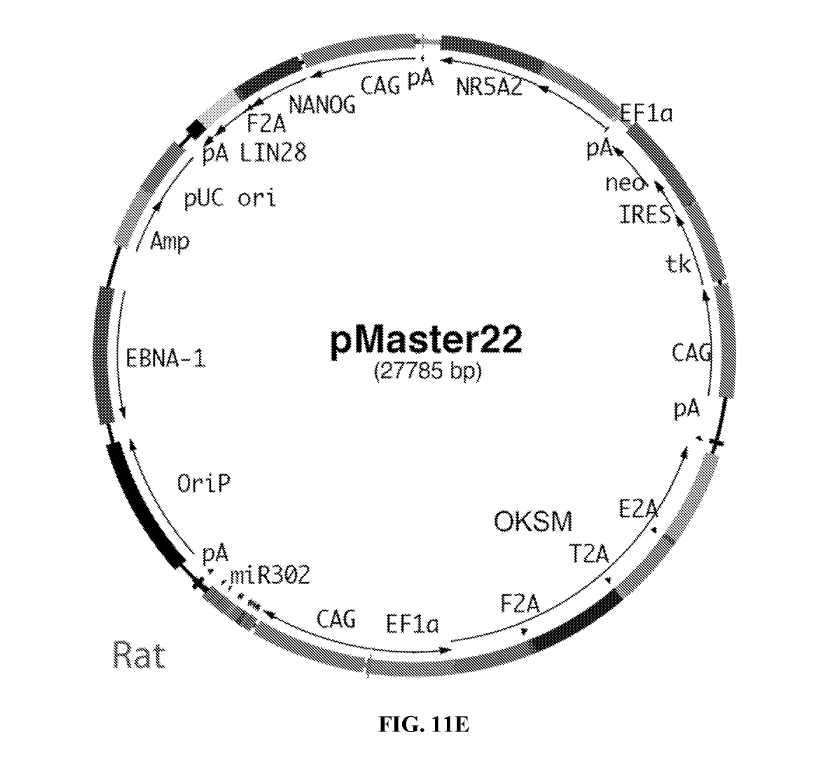

Although cells derived using all three pMaster vectors exhibited germ line potential, there were differences among them. Transgene-free iPS clones were not obtained using the pMaster1 vector, and all FIAU-selected pMaster1 clones examined retained the 6 reprogramming factors (OKSM, NANOG, and LIN28). Clones derived using pMaster3 tended to retain as least one reprogramming gene. However, the majority of pMaster12-derived clones were transgene-free. To further explore differences among iPS cell lines derived by these three vectors, the transcriptome of pMaster iPS clones grown in 2i medium and derived from each vector were compared with the transcriptome of the G4 ES cells also grown in 2i medium. Differentially expressed genes were again functionally annotated through GO terms and KEGG pathways (FIGS. 8 and 9). From pMaster1, to pMaster3, to pMaster12, upregulated genes in the resulting iPS cells were enriched for fewer and fewer GO and KEGG terms associated with cell differentiation and organ development (FIG. 9). Compared to pMaster1, downregulated genes in pMaster3- and pMaster12-derived iPS cells were enriched for fewer GO and KEGG terms associated with immune function and metabolic processes (FIG. 9). These results were consistent with pMaster12 iPS cells being more similar to the ES cells with respect to their gene expression profiles. From pMaster1 to pMaster3, the addition of NR5A2 gene in the pMaster3 vector allowed for the complete removal of transgenes. The NR5A2 gene improved pluripotency. To further dissect the potential role of NR5A2 gene, ES cells were grouped with pMaster3 derived iPS cells, and this group was compared with the pMaster1 derived cells. About 70 genes were observed to be differentially (>2 fold) expressed (FIG. 10). These differentially expressed genes may be used to further increase the reprogramming efficiency and iPS cell quality.

To test the broad applicability of the pMaster methods, pMaster vectors were constructed that each contained a species-specific miR302/367 gene cluster. Introduction of species-specific episomes into species-matched fibroblasts of rat and bat generated iPS cells at an efficiency of about 1%. Transgene-free clones for these two species were obtained. This result indicated that pMaster vectors also generated transgene free iPS cells for other species.

TABLE 1
Blastocyst and 8-cell injection results of iPS cell lines derived with
pMaster1 episomal vector.

| | | | | | Number of | |
|---|---|---|---|---|---|---|
| | | | | Injected | chimeras | |
| iPS cell | Transgene | Culture | Injected | 8-cell | and | Germ line |
| line | status | Media | blastocysts | embryos | chimerism | transmission |
| iPS322- | Partial | ES | 21 | 22 | 0 | — |
| 38s, ♂ | removal | | | | | |
| iPS322- | Partial | ES | 10 | 7 | 0 | — |
| 31p, ♂ | removal | | | | | |
| iPS322- | Partial | ES | 15 | 28 | 1 ♀ (8-cell | No |
| 40t, ♂ | removal | | | | injection), | |
| | | | | | 10% | |
| iPS322- | Partial | ES | 3 | 28 | 1 ♀ (8-cell | No |
| 45v, ♂ | removal | | | | injection), | |
| | | | | | 15% | |
| iPS322- | Partial | ES | 14 | 13 | 1 ♂ | No |
| 45w, ♂ | removal | | | | (blastocyst | |
| | | | | | injection), | |
| | | | | | 80% | |

TABLE 2
Blastocyst injection results of iPS cell lines
derived with pMaster series episomal vectors.

| | | | | Number of | |
|---|---|---|---|---|---|
| | Transgene | Culture | Number of | Chimeras and | Germ line |
| iPS cell line | status | Media | blastocysts | chimerism | transmission |
| iPS322-38s, ♂ | Partial | 2i | 90 | 1♂, 99% | Yes |
| (pMaster1) | removal | | | | |
| iPS322-40t, ♂ | Partial | 2i | 206 | 2♂, <2%; | Did not |
| (pMaster1) | removal | | | 3♀, 5% | breed |
| iPS344F28, ♂ | Partial | 2i | 189 | 2♂, 20%, 15% | Yes |
| (pMaster1) | removal | | | | |
| iPS344F30, ♂ | Partial | 2i | 234 | 4♂, 70%, 30%, | Yes |
| (pMaster1) | removal | | | 15%, 10%; | |
| | | | | 1♀, 50% | |
| iPS466F38, ♂ | NR5A2 | 2i | 444 | 10♂, 95%, 90%, | Yes |
| (pMaster3) | | | | 3 @70% etc | |
| | | | | 4♀, 60%, 50% etc | |
| iPS466F46, ♂ | Free | 2i | 378 | 6♂, 2@75%, 70% | Yes |
| (pMaster3) | | | | 40%, 30%, 10% | |
| | | | | 8♀, 90%, 75% etc | |
| iPSZX11-18-1, | Free | 2i | 235 | 8♂: 2@80%, | No |
| ♀ | | | | 2@40%, 1@30%, | |
| (pMaster12) | | | | 1@25%, 2@10% | |
| | | | | 9♀: 1@90%, | |
| | | | | 2@80%, 1@70%, | |
| | | | | 1@50%, 1@40%, | |
| | | | | 1@30%, 1@20%, | |
| | | | | 1@10% | |
| iPSZX11-18-2, | Free | 2i | 277 | ♂: 1@80%, | Yes |
| ♀ | | | | 2@30%, 2@25%, | |
| (pMaster12) | | | | 2@20%, 1@5%, | |
| | | | | 1@2% | |
| | | | | ♀: 1@75%, | |
| | | | | 1@70%, 3@50%, | |
| | | | | 2@40%, 2@30%, | |
| | | | | 1@25%, 1@20% | |

TABLE 3
List of oligos.

| | PCR | |
|---|---|---|
| Primer | amplified | |
| Pair | region | Oligo sequences |
| 1 | Neo-IRES | WS1384: ATGATGGATACTTTCTCGGCAGGA (SEQ ID |
| | | NO: 8) |
| | | WS1385: TGCCACGTTGTGAGTTGGATAGTT (SEQ ID |
| | | NO: 9) |
| | | Product size: 588 bp |
| 2 | Neo-IRES | W51386: GGACAGGTCGGTCTTGACAAAAAG (SEQ ID |
| | | NO: 10) |
| | | W51387: TCTGTTGAATGTCGTGAAGGAAGC (SEQ ID |
| | | NO: 11) |
| | | Product size: 569 bp |
| 3 | tk | WS1388: AATCCAGGATAAAGACGTGCATGG (SEQ ID |
| | | NO: 12) |
| | | WS1389: GACAATCGCGAACATCTACACCAC (SEQ ID |
| | | NO: 13) |
| | | Product size: 701 bp |
| 4 | tk | WS1390: ATACCGCACCGTATTGGCAAGTAG (SEQ ID |
| | | NO: 14) |
| | | WS1391: ACGTACCCGAGCCGATGACTTACT (SEQ ID |
| | | NO: 15) |
| | | Product size: 506 bp |
| 5 | MYC-SOX2 | WS1392: GAAGTTCTCCTCCTCGTCGCAGTA (SEQ ID |
| | | NO: 16) |
| | | WS1393: CCTGCAGTACAACTCCATGACCAG (SEQ ID |
| | | NO: 17) |
| | | Product size: 502 bp |
| 6 | MYC-SOX2 | WS1394: GTCGCAGATGAAACTCTGGTTCAC (SEQ ID |
| | | NO: 18) |
| | | WS1395: TGTGGTTACCTCTTCCTCCCACTC (SEQ ID |
| | | NO: 19) |
| | | Product size: 616 bp |
| 7 | SOX2-KLF4 | WS1396: TTCTCCGTCTCCGACAAAAGTTTC (SEQ ID |
| | | NO: 20) |
| | | WS1397: AAGAGTTCCCATCTCAAGGCACAC (SEQ ID |
| | | NO: 21) |
| | | Product size: 536 bp |
| 8 | SOX2-KLF4 | WS1398: CCTTCTTCATGAGCGTCTTGGTTT (SEQ ID |
| | | NO: 22) |
| | | WS1399: TGAACTGACCAGGCACTACCGTAA (SEQ ID |
| | | NO: 23) |
| | | Product size: 545 bp |
| 9 | KLF4-OCT4 | WS1400: GATCGTTGAACTCCTCGGTCTCTC (SEQ ID |
| | | NO: 24) |
| | | WS1401: ATGTGGTCCGAGTGTGGTTCTGTA (SEQ ID |
| | | NO: 25) |
| | | Product size: 618 bp |
| 10 | KLF4-OCT4 | WS1402: GGGTCAGCGAATTGGAGAGAATAA (SEQ ID |
| | | NO: 26) |
| | | WS1403: GATCAAGCAGCGACTATGCACAAC (SEQ ID |
| | | NO: 27) |
| | | Product size: 612 bp |
| 11 | oriP | WS1404: ATGGCTATGGGCAACACATAATCC (SEQ ID |
| | | NO: 28) |
| | | WS1405: CTCTCAGCGACCTCGTGAATATGA (SEQ ID |
| | | NO: 29) |
| | | Product size: 523 bp |
| 12 | oriP | WS1406: CACAAACCCCTTGGGCAATAAATA (SEQ ID |
| | | NO: 30) |
| | | WS1407: CCATTAGTGGTTTTGTGGGCAAGT (SEQ ID |
| | | NO: 31) |
| | | Product size: 500 bp |
| 13 | EBNA1 | WS1408: CCTCATCTCCATCACCTCCTTCAT (SEQ ID |
| | | NO: 32) |
| | | WS1409: TCCAACCCGAAATTTGAGAACATT (SEQ ID |
| | | NO: 33) |
| | | Product size: 487 bp |
| 14 | EBNA1 | WS1410: GGAAACCAGGGAGGCAAATCTACT (SEQ ID |
| | | NO: 34) |
| | | WS1411: TCACGTAGAAAGGACTACCGACGA (SEQ ID |
| | | NO: 35) |
| | | Product size: 357 bp |
| 15 | NANOG- | WS1416: AACCCTTCCATGTGCAGCTTACTC (SEQ ID |
| | LIN28 | NO: 36) |
| | | WS1417: CTGCTGGGGAAGGCCTTAATGTA (SEQ ID |
| | | NO:37) |
| | | Product size: 436 bp |
| 16 | NANOG- | W51418: CGCCTCTCACTCCCAATACAGAAT (SEQ ID |
| | LIN28 | NO: 38) |
| | | W51419: GAAGTGGCGTGAAACAGACTTTGA (SEQ ID |
| | | NO: 39) |
| | | Product size: 442 bp |
| 17 | NR5A2 | WS1555: TGTCAATTTGGCAGTTCTGGTTTT (SEQ ID |
| | | NO: 40) |
| | | WS1552: AGGGGTTTTATGCGATGGAGTTTC (SEQ ID |
| | | NO: 41) |
| | | Product size: 449 bp |
| 18 | NR5A2 | WS1556: GGACAACGCTTTCTCTGTGTTTTG (SEQ ID |
| | | NO: 42) |
| | | WS1554: CCAGCTTGGCACTTGATGTAATTC (SEQ ID |
| | | NO: 43) |
| | | Product size: 415 bp |
| 19 | EF1a-OCT4 | WS1551: GCACTAGCCCCACTCCAACCT (SEQ ID |
| | | NO: 44) |
| | | WS1552: AGGGGTTTTATGCGATGGAGTTTC (SEQ ID |
| | | NO: 45) |
| | | Product size: 445 bp |
| 20 | EF1a-OCT4 | WS1553: GAGTTGCTCTCCACCCCGACT (SEQ ID |
| | | NO: 46) |
| | | WS1554: CCAGCTTGGCACTTGATGTAATTC (SEQ ID |
| | | NO: 47) |
| | | Product size: 453 bp |
| 21 | Mir302/367 | WS1645: ATGGTGATGGATTGTTAAGCCAATG (SEQ ID |
| | cluster | NO: 48) |
| | | WS1646: TGGACAAACCACAACTAGAATGCAG (SEQ |
| | | ID NO: 49) |
| | | Product size: 304 bp |
| 22 | GFP | WS211: AAGCTGACCCTGAAGTTCATCTGC (SEQ ID |
| | | NO: 50) |
| | | WS212: CTTCTGCTTGTCGGCCATGATATAG (SEQ ID |
| | | NO: 51) |
| | | Product size: 354 bp |

### Example 3

### Summary of Results

As described above in Examples 1 and 2, assemblies of reprogramming factors and selection markers incorporated into single plasmids as non-integrating episomes were generated and employed to create germ line competent iPS cells. This single-episome system mediated reprogramming of somatic cells to pluripotent iPS cells. The above studies demonstrated that the inclusion of additional reprogramming factors provided in pMaster3 and in pMaster12 improved the probability of the reprogrammed cell gaining independence from the exogenous episomal genes to maintain pluripotency. Also, growth in 2i medium, compared to normal ES cell medium, improved the ability of the newly generated iPS cell to contribute to germ line formation. In particular, the pMaster12 yielded transgene-free iPS cells that, when grown in 2i medium, recapitulated mouse ES cells, in terms of their competency for generating germ line chimeras.

Additionally, the described methodology was applicable for the generation of iPS cells from multiple species including mouse, rat, and bat. As such, the above-described pMaster vectors may allow for the production of germ line-competent transgene-free iPS cells to use as surrogates for ES cells in those species for which authentic ES cell lines have yet to be developed.

In summary, the above-described studies demonstrated the generation of transgene-free iPS cells from fibroblasts and the production of healthy germ line chimeras from these iPS cell lines. Indeed we showed that cell lines generated by the PB vector system failed this most stringent test even after passing all the typical in vitro tests for transgene free iPS cells.

### 7. CLAUSES

Clause 1. An episome comprising OCT4, KLF4, SOX2, cMYC, NANOG, LIN28, and NR5A2.

Clause 2. The episome of clause 1, wherein at least two of OCT4, KLF4, SOX2, cMYC, NANOG, LIN28, and NR5A2 form a polycistronic locus expressed from a single promoter.

Clause 3. The episome of clause 2, further comprising a sequence encoding a 2A peptide between adjacent members of the polycistronic locus.

Clause 4. The episome of clause 1, further comprising EBNA-1/oriP.

Clause 5. The episome of clause 1, further comprising the microRNA 302/367 gene cluster.

Clause 6. The episome of clause 1, further comprising a positive selection marker.

Clause 7. The episome of clause 6, wherein the positive selection marker is neomycin resistance.

Clause 8. The episome of clause 1, further comprising a negative selection marker.

Clause 9. The episome of clause 8, wherein the negative selection marker is HSV-tk.

Clause 10. A method for producing an induced pluripotent stem (iPS) cell, comprising: (a) introducing an episome into a cell, wherein the episome comprises OCT4, KLF4, SOX2, cMYC, NANOG, LIN28, and NR5A2; (b) growing the cell under conditions to select for the presence of the episome; (c) selecting a primary clone; and (d) growing the primary clone in a medium comprising a MEK inhibitor and a GSK3b inhibitor.

Clause 11. The method of clause 10, wherein the cell is a fibroblast.

Clause 12. The method of clause 10, wherein the episome further comprises the microRNA 302/367 gene cluster.

Clause 13. The method of clause 10, further comprising: growing the primary clone under conditions to select for the absence of the episome, selecting a secondary clone, and growing the secondary clone in a medium comprising a MEK inhibitor and a GSK3b inhibitor.

Clause 14. The method of clause 10, wherein the medium comprises a MEK inhibitor and a GSK3b inhibitor and is serum free.

Clause 15. The method of clause 10, further comprising: verifying that the primary clone is transgene-free.

Clause 16. The method of clause 12, wherein the efficiency of iPS generation is at least about 0.2%.

Clause 17. The method of clause 10, wherein the resulting iPS cells exhibit germ line competence.

Clause 18. An iPS cell produced according to the method of clause 10.

Clause 19. The iPS cell of claim 18, wherein the iPS cell is transgene-free.

Clause 20. The iPS cell of claim 18, wherein the iPS cell exhibits germ line competence.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. An episome comprising OCT4, KLF4, SOX2, cMYC, NANOG, LIN28, and NR5A2.

2. The episome of claim 1, wherein at least two of OCT4, KLF4, SOX2, cMYC, NANOG, LIN28, and NR5A2 form a polycistronic locus expressed from a single promoter.

3. The episome of claim 2, further comprising a sequence encoding a 2A peptide between adjacent members of the polycistronic locus.

4. The episome of claim 1, further comprising EBNA-1/oriP.

5. The episome of claim 1, further comprising the microRNA 302/367 gene cluster.

6. The episome of claim 1, further comprising a positive selection marker.

7. The episome of claim 6, wherein the positive selection marker is neomycin resistance.

8. The episome of claim 1, further comprising a negative selection marker.

9. The episome of claim 8, wherein the negative selection marker is HSV-tk.

10. A method for producing an induced pluripotent stem (iPS) cell, comprising:
(a) introducing an episome into a cell, wherein the episome comprises OCT4, KLF4, SOX2, cMYC, NANOG, LIN28, and NR5A2;
(b) growing the cell under conditions to select for the presence of the episome;
(c) selecting a primary clone; and
(d) growing the primary clone in a medium comprising a MEK inhibitor and a GSK3b inhibitor.

11. The method of claim 10, wherein the cell is a fibroblast.

12. The method of claim 10, wherein the episome further comprises the microRNA 302/367 gene cluster.

13. The method of claim 10, further comprising: growing the primary clone under conditions to select for the absence of the episome, selecting a secondary clone, and growing the secondary clone in a medium comprising a MEK inhibitor and a GSK3b inhibitor.

14. The method of claim 10, wherein the medium comprises a MEK inhibitor and a GSK3b inhibitor and is serum free.

15. The method of claim 10, further comprising: verifying that the primary clone is transgene-free.

16. The method of claim 12, wherein the efficiency of iPS generation is at least about 0.2%.

17. The method of claim 10, wherein the resulting iPS cells exhibit germ line competence.

18. An iPS cell produced according to the method of claim 10.

19. The iPS cell of claim 18, wherein the iPS cell is transgene-free.

20. The iPS cell of claim 18, wherein the iPS cell exhibits germ line competence.
